(19) United States
(12) Patent Application Publication
(10) Pub. No.: US 2021/0050090 A1
(43) Pub. Date: Feb. 18, 2021

(54) SMOKING CESSATION THERAPEUTIC SYSTEM AND REMOTE PATIENT MONITORING DEVICE

(71) Applicant: Click Therapeutics, Inc., New York, NY (US)

(72) Inventors: DAVID KLEIN, New York, NY (US); VICTOR GAO, Dix Hills, NY (US); JOSHUA STEINERMAN, Bala Cynwyd, PA (US)

(21) Appl. No.: 17/085,828

(22) Filed: Oct. 30, 2020

Related U.S. Application Data
(63) Continuation of application No. 15/563,477, filed on Sep. 29, 2017.
(60) Provisional application No. 62/142,374, filed on Apr. 2, 2015.
(60) Provisional application No. 62/159,172, filed on May 8, 2015.
(60) Provisional application No. 62/217,524, filed on Sep. 11, 2015.

(51) Int. Cl. G16H 20/70; G16H 40/67

(52) U.S. Cl. CPC G16H 20/70; G16H 10/60; G16H 40/67

(57) ABSTRACT

The present disclosure relates to a smoking cessation therapeutic system and remote patient monitoring device. The smoking cessation therapeutic system is configured to communicate with a plurality of remote patient monitoring devices to deliver computer-executable instructions to the remote patient monitoring devices to provide digital therapeutics to patients of the patient monitoring devices to facilitate smoking cessation among the patients. The digital therapeutics are updated by the system responsive to the patient's response to the previously presented digital therapeutics.

### CROSS-REFERENCE TO RELATED APPLICATIONS

This application is a continuation of and claims priority to and the benefit of U.S. patent application Ser. No. 15/563,477, filed on Sep. 29, 2017, which is a National Stage Entry of and claims priority to and the benefit of International Patent Application No. PCT/US2016/025860, filed on Apr. 4, 2016, which claims priority to U.S. and the benefit of Provisional Patent Application No. 62/142,374 filed on Apr. 2, 2015, U.S. Provisional Patent Application No. 62/159,172 filed on May 8, 2015, and U.S. Provisional Patent Application No. 62/217,524 filed on Sep. 11, 2015, each of which are herein incorporated by reference in their entirety.

### BACKGROUND

Each year millions of people resolve to quit smoking. However, there are very few non-chemical options for quitting smoking. Current mobile technologies promoting smoking cessation are limited to informational applications (i.e. reference materials for quitting smoking, such as general health information) and messaging applications that send text messages or other messages to users encouraging them to quit smoking.

### SUMMARY

According to one aspect of the disclosure, a smoking cessation therapeutic system includes a computing device that includes one or more processors. The smoking cessation therapeutic system also includes a smoking cessation application that is stored in the memory and executable by the one or more processors. The smoking cessation application is configured to decouple sensorimotor experiences of smoking experienced by a user from non-nicotinic reinforcers through initiation of a breathing exercise. The smoking cessation application can include a breathing sound effects component. The breathing sound effects component is configured to output, via an audio output of a computing device, such as a patient monitoring device, one or more breathing sound effects. The breathing sound effects can correspond to a breathing pace representative of controlled breathing for a duration of the breathing exercise. The breathing sound effects including an inhaling sound effect and an exhaling sound effect. The smoking cessation application also includes a digital cigarette component. The digital cigarette component is configured to display, on a display of the computing device, a digital cigarette. The digital cigarette extends along a length of the display. The digital cigarette includes a top portion that includes a lit portion and a bottom portion that includes a cigarette butt of the digital cigarette. The smoking cessation application also includes a haptic control component to provide sensorimotor engagement. The haptic control component is configured to provide a haptic control bar that extends horizontally across the digital cigarette displayed on the client device. The haptic control bar is configured to be guided along a predefined axis extending along a length of the digital cigarette to train the user to haptically follow the haptic control bar while the haptic control bar slides from the lit portion towards the cigarette butt and from the cigarette butt to the lit portion. The smoking cessation application 250 also includes a breath capacity representation component that is configured to provide a graphical representation of breath capacity. The breath capacity representation component changes appearance based on a movement of the haptic control bar. The graphical representation of breath capacity has a first appearance when the haptic control bar is positioned at the lit portion and a second appearance when the haptic control bar is positioned at the cigarette butt. The breathing exercise, via the smoking cessation application, includes providing non-nicotinic stimuli through a combination of moving the haptic control bar along the axis of the digital cigarette in synchrony with the breathing sound effects.

In some implementations, the smoking cessation application includes a timer component that can be configured to provide a timer that indicates an amount of time lapsed from a beginning of the initiation of the breathing exercise or an amount of time remaining until an end of the breathing exercise. The haptic control bar can slide between the lit portion and the cigarette butt at a speed corresponding to the breathing pace. The haptic control bar can slide from the lit portion towards the cigarette butt at the same time the inhaling sound effect is output by the breathing sound effects component. The haptic control bar can slide from the cigarette butt to the lit portion at the same time the exhaling sound effect is output by the breathing sound effects component.

In some implementations, the graphical representation of breath capacity can include an image of at least one lung. The appearance of the at least one lung can be based on a position of the haptic control bar relative to at least one of the cigarette butt or the lit portion. In some implementations, the smoking cessation application can include a notification component that is configured to generate and deliver messages to the user at predetermined times and receive, from the client device, feedback on at least one message of the delivered messages indicating an efficacy of the message.

According to another aspect of the disclosure, a system includes a computing device that includes one or more processors. The system also includes a smoking cessation application stored in a memory and executable by the one or more processors. The smoking cessation application includes a sensorimotor decoupling component that is configured to decouple sensorimotor experiences of a smoking experienced by a user from non-nicotinic reinforcers through initiation of a breathing exercise. The sensorimotor decoupling component includes a breathing component that is configured to provide a breathing indicator. The breathing indicator corresponds to a breathing pace and is displayed for a duration of the breathing exercise. The breathing indicator includes a first effect representative of an inhaling phase and a second effect representative of an exhaling phase. The sensorimotor decoupling component includes a haptic control component to provide sensorimotor engagement. The haptic control component is configured to provide a haptic control bar. The haptic control bar is configured to be guided along a predefined axis to train the user to haptically follow the haptic control bar along the predefined axis. The sensorimotor decoupling component also includes a breath capacity representation component that is configured to provide a graphical representation of breath capacity. The graphical representation changes appearance based on a movement of the haptic control bar. The graphical representation of breath capacity has a first appearance when the haptic control bar is positioned at a first position along the predefined axis and a second appearance when the haptic control bar is positioned at a second position along the predefined axis. The initiation of the breathing exercise via the smoking cessation application includes providing non-nicotinic stimuli through a combination of moving the haptic control bar along the predefined axis in synchrony with the breathing indicator. The sensorimotor decoupling component also includes a notification component that is configured to generate and deliver messages to the patient monitoring device at predetermined times and receive, from the patient monitoring device, feedback messages on at least one message of the delivered messages indicating an efficacy of the message. The sensorimotor decoupling component also includes a metrics component that is configured to calculate a total number of cigarettes avoided, a financial savings value, and a lifespan adjustment value by the user based on performing the breathing exercise.

In some implementations, the smoking cessation application includes a timer component that is configured to provide a timer that displays an amount of time lapsed from a beginning of the initiation of the breathing exercise or an amount of time remaining until an end of the breathing exercise. The haptic control bar can slide between the first position and the second position at a speed corresponding to the breathing pace. In some implementations, the haptic control bar slides from the first position towards the second position at the same time the first effect is output by the breathing component. In some implementations, the haptic control bar slides from the second position towards the first position at the same time the second sound effect is output by the breathing component.

The graphical representation of the breath capacity can include an image of at least one lung. An appearance of the at least one lung can be based on a position of the haptic control bar relative to at least one of the first position or the second position. The smoking cessation application can include a timer component that is configured to provide a timer indicating an amount of time lapsed from a beginning of the initiation of the breathing exercise or provide an amount of time remaining until an end of the breathing exercise.

In some implementations, the financial saving value is calculated based on a product of the number of cigarettes avoided and a price of a cigarette. The lifespan adjustment value can be calculated based on a number of cigarettes avoided. The notification component can identify an anticipated craving window of the user and deliver at least one message of the messages to the user responsive to identifying the anticipated craving window.

According to another aspect of the disclosure, a method includes executing, by a computing device including one or more processors, a smoking cessation application. The smoking cessation application is configured to decouple sensorimotor experiences of smoking experienced by a user from non-nicotinic reinforcers through initiation of a breathing exercise. The method includes outputting, by a breathing sound effects component of the smoking cessation application, one or more breathing sound effects that correspond to a breathing pace representative of controlled breathing. The sound effects are output for a duration of the breathing exercise. The breathing sound effects including an inhaling sound effect and an exhaling sound effect. The method also includes displaying, by a digital cigarette component of the smoking cessation application, on a display of the computing device, a digital cigarette that extends along a length of the display. The digital cigarette includes a top portion with a lit portion of the digital cigarette and a bottom portion with a cigarette butt of the digital cigarette. The method also includes providing, by a haptic control component of the smoking cessation application to provide sensorimotor engagement, a haptic control bar extending horizontally across the digital cigarette. The haptic control bar is configured to be guided along a predefined axis extending along a length of the digital cigarette to train the user to haptically follow the haptic control bar while the haptic control bar slides from the lit portion towards the cigarette butt and from the cigarette butt to the lit portion. The method also includes providing, by a breath capacity representation component of the smoking cessation application, a graphical representation of breath capacity. The graphical representation changes appearance based on a movement of the haptic control bar. The graphical representation of breath capacity has a first appearance when the haptic control bar is positioned at the lit portion and has a second appearance when the haptic control bar is positioned at the cigarette butt. Initiation of the breathing exercise via the smoking cessation application includes providing non-nicotinic stimuli through a combination of moving the haptic control bar along the axis of the digital cigarette in synchrony with the breathing sound effects.

In some implementations, executing the smoking cessation application can include providing, for display, a timer that indicates an amount of time lapsed from a beginning of the initiation of the breathing exercise or an amount of time remaining until an end of the breathing exercise. The haptic control bar can slide between the lit portion and the cigarette butt at a speed corresponding to the breathing pace. The haptic control bar can slide from the lit portion towards the cigarette butt at the same time the inhaling sound effect is output by the breathing sound effects component. The haptic control bar can slide from the cigarette butt to the lit portion at the same time the exhaling sound effect is output by the breathing sound effects component. In some implementations, the graphical representation of breath capacity includes an image of at least one lung. An appearance of the at least one lung is based on a position of the haptic control bar relative to at least one of the cigarette butt or the lit portion.

According to another aspect of the disclosure, a medical treatment system includes one or more processors and a memory having computer-executable instructions stored thereon. The system also includes a communications interface that is configured to establish communication channels with a plurality of remote patient monitoring devices. Each of the patient monitoring devices can correspond to a patient and is associated with a patient profile. The system also includes a patient profile type identification module that can include computer-executable instructions, which when executed by the one or more processors, causes the processors to determine a profile type of a patient based on data received from the patient monitoring device of the patient. The data can include query responses. The system can also include a smoking cessation treatment generation module including computer-executable instructions, which when executed by the one or more processors, causes the processors to generate a smoking cessation treatment plan corresponding to the patient. The smoking cessation treatment plan can include a plurality of events to be delivered to the patient monitoring device of the patient. Each event of the plurality of predetermined events can be generated based on the patient profile and in response to previous feedback received from the patient monitoring device of the patient. The system can also include an event delivery module including computer-executable instructions, which when executed by the one or more processors, causes the processors to deliver the event of the plurality of predetermined events corresponding to an event delivery schedule according to which the event is to be delivered to the patient monitoring device of the patient. The event including data, which when received by the patient monitoring device, causes an application executing on the patient monitoring device to display the event on a display of the patient monitoring device and record activity performed on the application executing on the patient monitoring device responsive to displaying the event. The system can also include an event feedback management module including computer-executable instructions, which when executed by the one or more processors, causes the processors to i) receive data identifying a status of the event from the patient monitoring device responsive to the event being displayed on the patient monitoring device, and ii) store, in the memory, the received data corresponding to each event. The smoking cessation treatment generation module can also be configured to modify the smoking cessation treatment plan responsive to the received data.

In some implementations, the received data can include an event identifier identifying the displayed event and a status identifier identifying the status of the event. The event feedback management module can be configured to determine a type of the displayed event based on the status identifier. The patient profile type can include one of an executive profile type and a motivational profile type. Each of the plurality of events can be one of a controlled breathing event, a social engagement event, a personalized message event, a treatment adherence event, a financial inventive event, and a digital diversion event. In some implementations, the smoking cessation treatment generation module is configured to modify the event delivery schedule.

According to another aspect of the disclosure a method includes receiving, via a communications interface of a smoking cessation therapeutics system including one or more processors configured to establish communication channels with a plurality of remote patient monitoring devices, a patient profile. The patient profile can correspond to a patient associated with one of the patient monitoring devices. The method can also include determining, via an identification module of the smoking cessation therapeutics system, a patient profile type of the patient based on data received from the patient monitoring device of the patient. The method can also include generating, by a smoking cessation treatment generation module of the smoking cessation therapeutics system, a smoking cessation treatment plan corresponding to the patient. The smoking cessation treatment plan can include a plurality of events to be delivered to the patient monitoring device of the patient. Each of the events of the plurality of predetermined events can be generated based on the patient profile and in response to previous feedback received from the patient monitoring device of the patient. The method can also include delivering, by an event delivery module of the smoking cessation therapeutics system, the plurality of predetermined events corresponding to an event delivery schedule according to which the event is to be delivered to the patient monitoring device of the patient. The event can include data, which when received by the patient monitoring device, causes an application executing on the patient monitoring device to display the event on a display of the patient monitoring device and record activity performed on the application executing on the patient monitoring device responsive to displaying the event. The method can also include receiving, an event feedback management module, data identifying a status of the event from the patient monitoring device responsive to the event being displayed on the patient monitoring device The method can also include storing, by the event feedback management module, received data corresponding to each event. The method can include modifying, by the smoking cessation treatment generation module, the smoking cessation treatment plan responsive to the received data.

In some implementations, the received data includes an event identifier identifying the displayed event and a status identifier identifying the status of the event, and wherein the event feedback management module is configured to determine a type of the displayed event. The patient profile type can include one of an executive profile type and a motivational profile type. Each of the plurality of events can be one of a controlled breathing event, a social engagement event, a personalized message event, a treatment adherence event, a financial inventive event, and a digital diversion event. In some implementations, the smoking cessation treatment generation module is further configured to modify the event delivery schedule.

The present disclosure describes various implementations of a smoking cessation therapeutic apparatus and remote patient monitoring device. The smoking cessation therapeutic apparatus can include a smoking cessation therapeutics system includes one or more computing devices that are configured to execute computer-readable instructions to bring about a behavioral change (for example, ceasing to smoke) in users of the smoking cessation therapeutics system. The smoking cessation therapeutics system can include one or more centralized servers configured to communicate with a plurality of computing devices, such as client devices. Users seeking to change their behavior can communicate with the smoking cessation therapeutics system via the client devices. These client devices can serve an input devices configured to receive input from the users. The input can be transmitted to the smoking cessation therapeutics system as network packets that include a payload carrying information of the user. In some implementations, the client devices can communicate with one or more other devices of the user, such as wearables, or other devices that may monitor, sense or otherwise interact with the user.

The smoking cessation therapeutics system can be configured to provide therapies and treatments to users. The therapies and treatments may include instructions for the user to perform particular missions or exercises. These therapies and treatments can link the digital world to the physical world. For instance, a therapy or treatment can include a breathing exercise. The smoking cessation therapeutics system can provide a user interface on the client device of the user inviting the user to perform a breathing exercise. In some implementations, the smoking cessation therapeutics system may be able to detect whether the user is performing the breathing exercise via one or more wearables attached to the user or via the client device itself. In some implementations, the smoking cessation therapeutics system may be unable to physically detect whether the user has performed the breathing exercise but may determine that the user has performed the breathing exercise by receiving an input from the user via the user interface of the client device indicating that the user performed the breathing exercise.

The smoking cessation therapeutics system applies evidence-based forms of intervention to bring about behavioral change in users. The smoking cessation therapeutics system provides tailored therapies at a personalized level based on one or more parameters of the user. The smoking cessation therapeutics system is adaptive and dynamic in that the smoking cessation therapeutics system is configured to adapt the treatments and therapies based on data received from the plurality of users. The smoking cessation therapeutics system can be configured to receive input and/or feedback from a plurality of users based on the therapies or treatments provided to the users. The feedback from the users can be used by the smoking cessation therapeutics system to modify or adjust therapies based on the feedback. In addition, the smoking cessation therapeutics system is configured to work with prescription drugs and over-the-counter therapies, configured either to promote treatment adherence or to develop and provide therapies and treatments to users to taper users' reliance on prescription drugs.

According to one aspect, the present disclosure is directed to methods and systems for a clinical smoking cessation therapeutics system. The smoking cessation therapeutic system uses an evidence-based mobile application to provide users who want to quit or change habits (for example, smoking) using a guided approach. The smoking cessation therapeutic system can deliver a personalized, tailored, adaptive and dynamic set of therapies, treatments, and activities to users to help the users identify neurological or other physiological changes representative of cravings and withdrawals, learn skills and techniques to manage situations involving cravings and withdrawals and build motivation to create and reinforce awareness and knowledge through conversations, visualizations of health benefits and progress, and community support. The smoking cessation therapeutic system also helps users develop coping skills for dealing with acute and chronic cravings and withdrawal symptoms. All along, via the application, the user is able to track their progress as they progress through the stages of changing a habit, namely, precontemplation, contemplation, preparation, action, and maintenance, termination, and in some instances relapse.

Aspects of the present disclosure also describe various implementations of a systems and method for identifying and predicting achievement of a behavioral endpoint using socialized financial incentives. In one aspect of the present disclosure, a method of using socialized financial incentives to affect a behavioral change endpoint can include receiving an indication of a desire to reach a behavior endpoint. The behavior endpoint can be the goal the user would like to reach or a habit the user would like to stop. The method can also include identifying an end date at which time the system determines whether the user reached the behavioral endpoint. The method also includes receiving, from a plurality of supporters, indications of financial support towards a fund that will be provided to the user if the user successfully reaches the endpoint. At the end date, the system implementing the method can receive a message or other form of indication, from each of the plurality of supporters, of whether the user reached the behavior endpoint. The system can process each of the received indications to determine whether the user reached the behavioral endpoint. If the system determines the user reached the endpoint, the system can initiate a transfer of the fund to the user.

### BRIEF DESCRIPTION OF THE FIGURES

The foregoing and other objects, aspects, features, and advantages of the disclosure will become more apparent and better understood by referring to the following description taken in conjunction with the accompanying drawings, in which:

FIG. 1A is a block diagram depicting an embodiment of a network environment comprising local devices in communication with remote devices.

FIGS. 1B-1D are block diagrams depicting embodiments of computers useful in connection with the methods and systems described herein.

FIG. 2 shows one embodiment of a smoking cessation system including a smoking cessation therapeutics system and a patient monitoring device communicatively coupled to the smoking cessation therapeutics system via a network.

FIG. 3A is a block diagram of one embodiment of the smoking cessation therapeutics system shown in FIG. 2.

FIG. 3B is a block diagram of one embodiment of a smoking cessation application executing on the patient monitoring device shown in FIG. 2.

FIG. 4A is an illustration of underlying data platform that includes application data collection flow, data analysis and hypothesis verification, and data utilization to create application behavior modification, application customization, and therapeutic feedback.

FIG. 4B is an illustration of multi-channel application data collection process with timestamp.

FIGS. 4C and 4D illustrate an event table storing data about events before and after updating the event table, respectively.

FIG. 5 is an illustration of an embodiment of the process for installing the application and performing initial setup.

FIG. 6A is an illustration of an embodiment of the visual and sonic stimulation for guided deep breathing aspect of the application.

FIG. 6B is an illustration of an embodiment of the data collection aspect of the application.

FIG. 7 is an illustration of an embodiment of the profile population of the application.

FIG. 8 is a graphical representation of a patient monitoring device interface illustrating a de-nicotinized virtual cigarette with haptic input guided deep breathing control bar, breath capacity representation and timer.

FIG. 9 is a graphical representation of a patient monitoring device interface displaying examples of automated tailored contextualized reinforcement text messages delivered to the patient monitoring device.

FIG. 10 is a graphical representation of a patient monitoring device interface representing reinforcing statistics calculated from individual user data with underlying formulas drawn from population data.

FIG. 11 is a graphical representation of a patient monitoring device interface illustrating a snapshot of a central dashboard that allows a user to access various functionalities and therapeutic features and keep track of quit journey.

FIG. 12 is an illustration of four different mechanisms of action the smoking cessation application is configured to provide.

FIG. 13 includes graphical representations of a patient monitoring device interface for controlled breathing including graphical representations relating to fresh air, mindful breathing, inspirations and social connections that serve as a craving wall.

FIG. 14 includes graphical representations of a patient monitoring device interface for personalized messaging as well as for displaying physiological visualizations of health recovery, progress towards becoming smoke-free via prizes and rewards and health metrics, including tools to fight and understand cravings.

FIG. 15 includes graphical representations of a patient monitoring device interface for social engagement.

FIG. 16 includes graphical representations of a patient monitoring device interface for treatment adherence options.

FIG. 17 includes graphical representations of a patient monitoring device interface for displaying various phases of the journey to quitting smoking.

FIG. 18 illustrates a block diagram of a method for decoupling sensorimotor experiences of smoking from reinforcers.

FIG. 19 is a block diagram of an example system for providing socialized financial incentives;

FIG. 20 is a flow diagram of an example method for providing socialized financial incentives using the system illustrated in FIG. 2;

FIGS. 21A-21G illustrate example user interfaces of applications and webpages for providing financial incentives to a user.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION OF THE DISCLOSURE

For purposes of reading the description of the various embodiments below, the following enumeration of the sections of the specification and their respective contents may be helpful:
Section A describes a network and computing environment which may be useful for practicing embodiments described herein; and
Section B describes embodiments of a smoking cessation therapeutic system and remote patient monitoring device.

### A. Network and Computing Environment

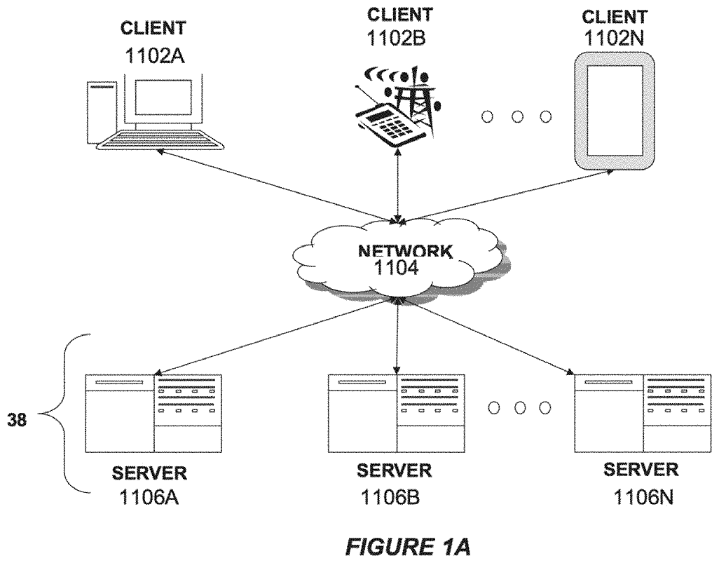

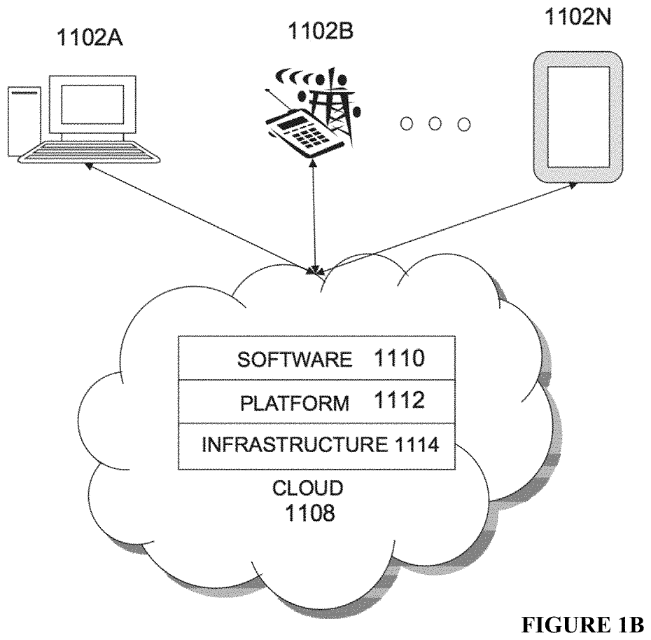

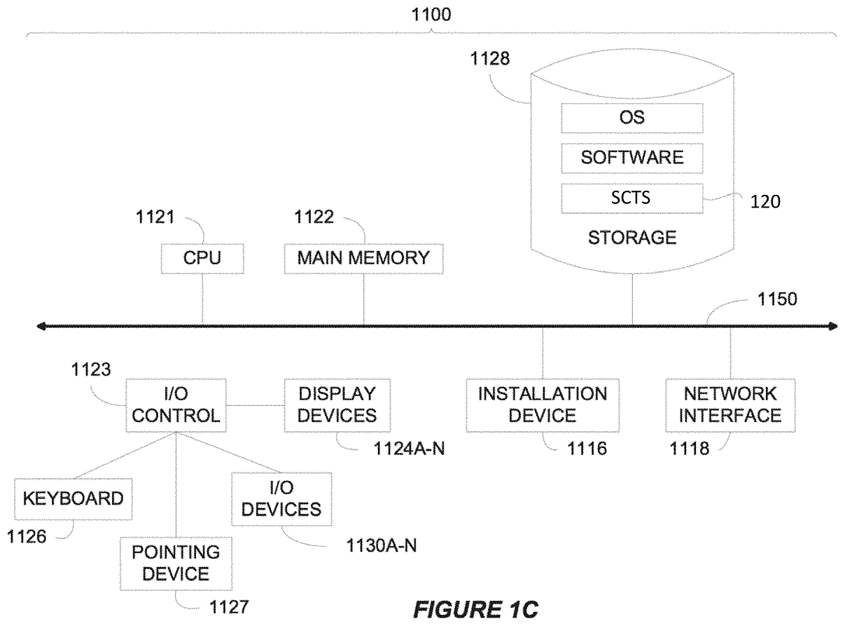

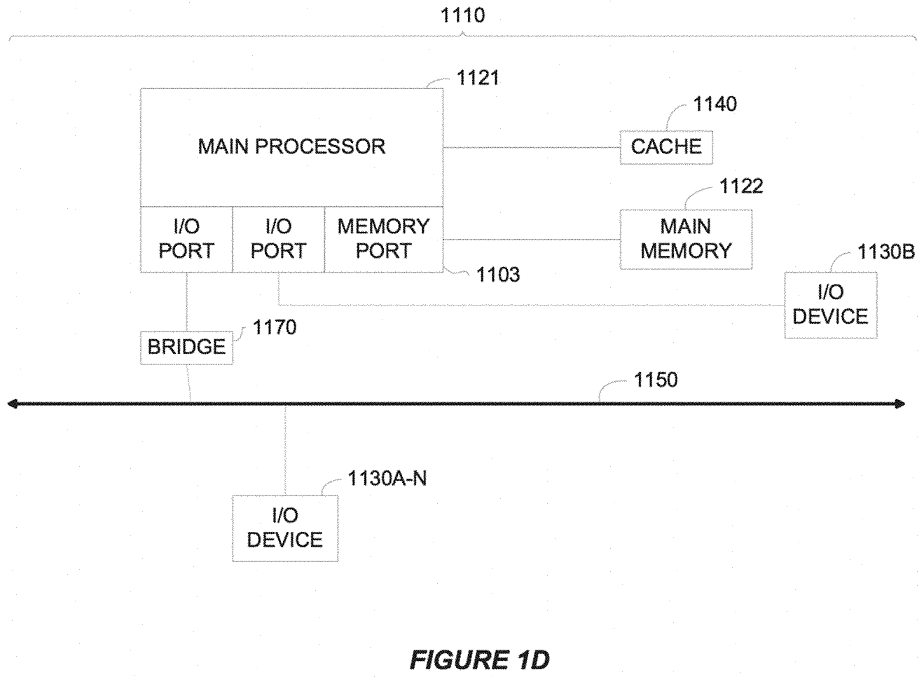

In addition to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In brief overview, the network environment includes one or more clients 1102a-1102n (also generally referred to as local machine(s) 1102, client(s) 1102, client node(s) 1102, client machine(s) 1102, client computer(s) 1102, client device(s) 1102, endpoint(s) 1102, or endpoint node(s) 1102) in communication with one or more servers 1106a-1106n (also generally referred to as server(s) 1106, node 1106, or remote machine(s) 1106) via one or more networks 1104. In some embodiments, a client 1102 has the capacity to function as both a client node seeking access to resources provided by a server and as a server providing access to hosted resources for other clients 1102a-1102n.

Although FIG. 1A shows a network 1104 between the clients 1102 and the servers 1106, the clients 1102 and the servers 1106 may be on the same network 1104. In some embodiments, there are multiple networks 1104 between the clients 1102 and the servers 1106. In one of these embodiments, a network 1104′ (not shown) may be a private network and a network 1104 may be a public network. In another of these embodiments, a network 1104 may be a private network and a network 1104′ a public network. In still another of these embodiments, networks 1104 and 1104′ may both be private networks.

The network 1104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, or 4G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 1G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 1104 may be any type and/or form of network. The geographical scope of the network 1104 may vary widely and the network 1104 can be a body area network (BAN), a personal area network (PAN), a local-area network (LAN), e.g. Intranet, a metropolitan area network (MAN), a wide area network (WAN), or the Internet. The topology of the network 1104 may be of any form and may include, e.g., any of the following: point-to-point, bus, star, ring, mesh, or tree. The network 1104 may be an overlay network which is virtual and sits on top of one or more layers of other networks 1104′. The network 1104 may be of any such network topology as known to those ordinarily skilled in the art capable of supporting the operations described herein. The network 1104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 1104 may be a type of a broadcast network, a telecommunications network, a data communication network, or a computer network.

In some embodiments, the system may include multiple, logically-grouped servers 1106. In one of these embodiments, the logical group of servers may be referred to as a server farm 38 or a machine farm 38. In another of these embodiments, the servers 1106 may be geographically dispersed. In other embodiments, a machine farm 38 may be administered as a single entity. In still other embodiments, the machine farm 38 includes a plurality of machine farms 38. The servers 1106 within each machine farm 38 can be heterogeneous—one or more of the servers 1106 or machines 1106 can operate according to one type of operating system platform (e.g., WINDOWS NT, manufactured by Microsoft Corp. of Redmond, Wash.), while one or more of the other servers 1106 can operate on according to another type of operating system platform (e.g., Unix, Linux, or Mac OS X).

In one embodiment, servers 1106 in the machine farm 38 may be stored in high-density rack systems, along with associated storage systems, and located in an enterprise data center. In this embodiment, consolidating the servers 1106 in this way may improve system manageability, data security, the physical security of the system, and system performance by locating servers 1106 and high performance storage systems on localized high performance networks. Centralizing the servers 1106 and storage systems and coupling them with advanced system management tools allows more efficient use of server resources.

The servers 1106 of each machine farm 38 do not need to be physically proximate to another server 1106 in the same machine farm 38. Thus, the group of servers 1106 logically grouped as a machine farm 38 may be interconnected using a wide-area network (WAN) connection or a metropolitan-area network (MAN) connection. For example, a machine farm 38 may include servers 1106 physically located in different continents or different regions of a continent, country, state, city, campus, or room. Data transmission speeds between servers 1106 in the machine farm 38 can be increased if the servers 1106 are connected using a local-area network (LAN) connection or some form of direct connection. Additionally, a heterogeneous machine farm 38 may include one or more servers 1106 operating according to a type of operating system, while one or more other servers 1106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, Calif.; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 1106 may comprise components, subsystems and modules to support one or more management services for the machine farm 38. In one of these embodiments, one or more servers 1106 provide functionality for management of dynamic data, including techniques for handling failover, data replication, and increasing the robustness of the machine farm 38. Each server 1106 may communicate with a persistent store and, in some embodiments, with a dynamic store.

Server 1106 may be a file server, application server, web server, proxy server, appliance, network appliance, gateway, gateway server, virtualization server, deployment server, SSL VPN server, or firewall. In one embodiment, the server 1106 may be referred to as a remote machine or a node. In another embodiment, a plurality of nodes 290 may be in the path between any two communicating servers.

Referring to FIG. 1B, a cloud computing environment is depicted. A cloud computing environment may provide client 1102 with one or more resources provided by a network environment. The cloud computing environment may include one or more clients 1102a-1102n, in communication with the cloud 1108 over one or more networks 1104. Clients 1102 may include, e.g., thick clients, thin clients, and zero clients. A thick client may provide at least some functionality even when disconnected from the cloud 1108 or servers 1106. A thin client or a zero client may depend on the connection to the cloud 1108 or server 1106 to provide functionality. A zero client may depend on the cloud 1108 or other networks 1104 or servers 1106 to retrieve operating system data for the client device. The cloud 1108 may include back end platforms, e.g., servers 1106, storage, server farms or data centers.

The cloud 1108 may be public, private, or hybrid. Public clouds may include public servers 1106 that are maintained by third parties to the clients 1102 or the owners of the clients. The servers 1106 may be located off-site in remote geographical locations as disclosed above or otherwise. Public clouds may be connected to the servers 1106 over a public network. Private clouds may include private servers 1106 that are physically maintained by clients 1102 or owners of clients. Private clouds may be connected to the servers 1106 over a private network 1104. Hybrid clouds 1108 may include both the private and public networks 1104 and servers 1106.

The cloud 1108 may also include a cloud based delivery, e.g. Software as a Service (SaaS) 1110, Platform as a Service (PaaS) 1112, and Infrastructure as a Service (IaaS) 1114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS include AMAZON WEB SERVICES provided by Amazon.com, Inc., of Seattle, Wash., RACKSPACE CLOUD provided by Rackspace US, Inc., of San Antonio, Tex., Google Compute Engine provided by Google Inc. of Mountain View, Calif., or RIGHTSCALE provided by RightScale, Inc., of Santa Barbara, Calif. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Wash., Google App Engine provided by Google Inc., and HEROKU provided by Heroku, Inc. of San Francisco, Calif. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, Calif., or OFFICE 365 provided by Microsoft Corporation. Examples of SaaS may also include data storage providers, e.g. DROPBOX provided by Dropbox, Inc. of San Francisco, Calif., Microsoft SKYDRIVE provided by Microsoft Corporation, Google Drive provided by Google Inc., or Apple ICLOUD provided by Apple Inc. of Cupertino, Calif.

Clients 1102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 1102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 1102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, Calif.). Clients 1102 may also access SaaS resources through smartphone or tablet applications, including, for example, Salesforce Sales Cloud, or Google Drive app. Clients 1102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 1102 and server 1106 may be deployed as and/or executed on any type and form of computing device, e.g. a computer, network device or appliance capable of communicating on any type and form of network and performing the operations described herein. FIGS. 1C and 1D depict block diagrams of a computing device 1100 useful for practicing an embodiment of the client 1102 or a server 1106. As shown in FIGS. 1C and 1D, each computing device 1100 includes a central processing unit 1121, and a main memory unit 1122. As shown in FIG. 1C, a computing device 1100 may include a storage device 1128, an installation device 1116, a network interface 1118, an I/O controller 1123, display devices 1124a-1124n, a keyboard 1126 and a pointing device 1127, e.g. a mouse. The storage device 1128 may include, without limitation, an operating system, software, and a software of a smoking cessation therapeutics system (SCTS) 120. As shown in FIG. 1D, each computing device 1100 may also include additional optional elements, e.g. a memory port 1103, a bridge 1170, one or more input/output devices 11200a-11200n (generally referred to using reference numeral 11200), and a cache memory 1140 in communication with the central processing unit 1121.

The central processing unit 1121 is any logic circuitry that responds to and processes instructions fetched from the main memory unit 1122. In many embodiments, the central processing unit 1121 is provided by a microprocessor unit, e.g.: those manufactured by Intel Corporation of Mountain View, Calif.; those manufactured by Motorola Corporation of Schaumburg, Ill.; the ARM processor and TEGRA system on a chip (SoC) manufactured by Nvidia of Santa Clara, Calif.; the POWER7 processor, those manufactured by International Business Machines of White Plains, N.Y.; or those manufactured by Advanced Micro Devices of Sunnyvale, Calif. The computing device 1100 may be based on any of these processors, or any other processor capable of operating as described herein. The central processing unit 1121 may utilize instruction level parallelism, thread level parallelism, different levels of cache, and multi-core processors. A multi-core processor may include two or more processing units on a single computing component. Examples of a multi-core processors include the AMD PHENOM IIX2, INTEL CORE i5 and INTEL CORE i7.

Main memory unit 1122 may include one or more memory chips capable of storing data and allowing any storage location to be directly accessed by the microprocessor 1121. Main memory unit 1122 may be volatile and faster than storage 1128 memory. Main memory units 1122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 1122 or the storage 1128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 1122 may be based on any of the above described memory chips, or any other available memory chips capable of operating as described herein. In the embodiment shown in FIG. 1C, the processor 1121 communicates with main memory 1122 via a system bus 1150 (described in more detail below). FIG. 1D depicts an embodiment of a computing device 1100 in which the processor communicates directly with main memory 1122 via a memory port 1103. For example, in FIG. 1D the main memory 1122 may be DRDRAM.

FIG. 1D depicts an embodiment in which the main processor 1121 communicates directly with cache memory 1140 via a secondary bus, sometimes referred to as a backside bus. In other embodiments, the main processor 1121 communicates with cache memory 1140 using the system bus 1150. Cache memory 1140 typically has a faster response time than main memory 1122 and is typically provided by SRAM, BSRAM, or EDRAM. In the embodiment shown in FIG. 1D, the processor 1121 communicates with various I/O devices 11200 via a local system bus 1150. Various buses may be used to connect the central processing unit 1121 to any of the I/O devices 11200, including a PCI bus, a PCI-X bus, or a PCI-Express bus, or a NuBus. For embodiments in which the I/O device is a video display 1124, the processor 1121 may use an Advanced Graphics Port (AGP) to communicate with the display 1124 or the I/O controller 1123 for the display 1124. FIG. 1D depicts an embodiment of a computer 1100 in which the main processor 1121 communicates directly with I/O device 11200b or other processors 1121′ via HYPERTRANSPORT, RAPIDIO, or INFINIBAND communications technology. FIG. 1D also depicts an embodiment in which local busses and direct communication are mixed: the processor 1121 communicates with I/O device 11200a using a local interconnect bus while communicating with I/O device 11200b directly.

A wide variety of I/O devices 11200a-11200n may be present in the computing device 1100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 11200a-11200n may include a combination of multiple input or output devices, including, e.g., Microsoft KINECT, Nintendo Wiimote for the WIT, Nintendo WII U GAMEPAD, or Apple IPHONE. Some devices 11200a-11200n allow gesture recognition inputs through combining some of the inputs and outputs. Some devices 11200a-11200n provides for facial recognition which may be utilized as an input for different purposes including authentication and other commands. Some devices 11200a-11200n provides for voice recognition and inputs, including, e.g., Microsoft KINECT, SIRI for IPHONE by Apple, Google Now or Google Voice Search.

Additional devices 11200a-11200n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 11200a-11200n, display devices 1124a-1124n or group of devices may be augment reality devices. The I/O devices may be controlled by an I/O controller 1123 as shown in FIG. 1C. The I/O controller may control one or more I/O devices, such as, e.g., a keyboard 1126 and a pointing device 1127, e.g., a mouse or optical pen. Furthermore, an I/O device may also provide storage and/or an installation medium 1116 for the computing device 1100. In still other embodiments, the computing device 1100 may provide USB connections (not shown) to receive handheld USB storage devices. In further embodiments, an I/O device 11200 may be a bridge between the system bus 1150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 1124a-1124n may be connected to I/O controller 1123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 1124a-1124n may also be a head-mounted display (HMD). In some embodiments, display devices 1124a-1124n or the corresponding I/O controllers 1123 may be controlled through or have hardware support for OPENGL or DIRECTX API or other graphics libraries.

In some embodiments, the computing device 1100 may include or connect to multiple display devices 1124a-1124n, which each may be of the same or different type and/or form. As such, any of the I/O devices 11200a-11200n and/or the I/O controller 1123 may include any type and/or form of suitable hardware, software, or combination of hardware and software to support, enable or provide for the connection and use of multiple display devices 1124a-1124n by the computing device 1100. For example, the computing device 1100 may include any type and/or form of video adapter, video card, driver, and/or library to interface, communicate, connect or otherwise use the display devices 1124a-1124n. In one embodiment, a video adapter may include multiple connectors to interface to multiple display devices 1124a-1124n. In other embodiments, the computing device 1100 may include multiple video adapters, with each video adapter connected to one or more of the display devices 1124a-1124n. In some embodiments, any portion of the operating system of the computing device 1100 may be configured for using multiple displays 1124a-1124n. In other embodiments, one or more of the display devices 1124a-1124n may be provided by one or more other computing devices 1100a or 1100b connected to the computing device 1100, via the network 1104. In some embodiments software may be designed and constructed to use another computer's display device as a second display device 1124a for the computing device 1100. For example, in one embodiment, an Apple iPad may connect to a computing device 1100 and use the display of the device 1100 as an additional display screen that may be used as an extended desktop. One ordinarily skilled in the art will recognize and appreciate the various ways and embodiments that a computing device 1100 may be configured to have multiple display devices 1124a-1124n.

Referring again to FIG. 1C, the computing device 1100 may comprise a storage device 1128 (e.g. one or more hard disk drives or redundant arrays of independent disks) for storing an operating system or other related software, and for storing application software programs such as any program related to the software for the smoking cessation therapeutics system 120. Examples of storage device 1128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 1128 may be non-volatile, mutable, or read-only. Some storage device 1128 may be internal and connect to the computing device 1100 via a bus 1150. Some storage device 1128 may be external and connect to the computing device 1100 via a I/O device 11200 that provides an external bus. Some storage device 1128 may connect to the computing device 1100 via the network interface 1118 over a network 1104, including, e.g., the Remote Disk for MACBOOK AIR by Apple. Some client devices 1100 may not require a non-volatile storage device 1128 and may be thin clients or zero clients 1102. Some storage device 1128 may also be used as an installation device 1116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 1100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 1102. An application distribution platform may include a repository of applications on a server 1106 or a cloud 1108, which the clients 1102a-1102n may access over a network 1104. An application distribution platform may include application developed and provided by various developers. A user of a client device 1102 may select, purchase and/or download an application via the application distribution platform.

Furthermore, the computing device 1100 may include a network interface 1118 to interface to the network 1104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 1100 communicates with other computing devices 1100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Fla. The network interface 1118 may comprise a built-in network adapter, network interface card, PCMCIA network card, EXPRESSCARD network card, card bus network adapter, wireless network adapter, USB network adapter, modem or any other device suitable for interfacing the computing device 1100 to any type of network capable of communication and performing the operations described herein.

A computing device 1100 of the sort depicted in FIGS. 1B and 1C may operate under the control of an operating system, which controls scheduling of tasks and access to system resources. The computing device 1100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Wash.; MAC OS and iOS, manufactured by Apple, Inc. of Cupertino, Calif.; and Linux, a freely-available operating system, e.g. Linux Mint distribution (“distro”) or Ubuntu, distributed by Canonical Ltd. of London, United Kingom; or Unix or other Unix-like derivative operating systems; and Android, designed by Google, of Mountain View, Calif., among others. Some operating systems, including, e.g., the CHROME OS by Google, may be used on zero clients or thin clients, including, e.g., CHROMEBOOKS.

The computer system 1100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 1100 has sufficient processor power and memory capacity to perform the operations described herein. In some embodiments, the computing device 1100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 1100 is a gaming system. For example, the computer system 1100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Wash.

In some embodiments, the computing device 1100 is a digital audio player such as the Apple IPOD, IPOD Touch, and IPOD NANO lines of devices, manufactured by Apple Computer of Cupertino, Calif. Some digital audio players may have other functionality, including, e.g., a gaming system or any functionality made available by an application from a digital application distribution platform. For example, the IPOD Touch may access the Apple App Store. In some embodiments, the computing device 1100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 1100 is a tablet e.g. the IPAD line of devices by Apple; GALAXY TAB family of devices by Samsung; or KINDLE FIRE, by Amazon.com, Inc. of Seattle, Wash. In other embodiments, the computing device 1100 is a eBook reader, e.g. the KINDLE family of devices by Amazon.com, or NOOK family of devices by Barnes & Noble, Inc. of New York City, N.Y.

In some embodiments, the communications device 1102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc; or a Motorola DROID family of smartphones. In yet another embodiment, the communications device 1102 is a laptop or desktop computer equipped with a web browser and a microphone and speaker system, e.g. a telephony headset. In these embodiments, the communications devices 1102 are web-enabled and can receive and initiate phone calls. In some embodiments, a laptop or desktop computer is also equipped with a webcam or other video capture device that enables video chat and video call.

In some embodiments, the status of one or more machines 1102, 1106 in the network 1104 is monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

### B. Smoking Cessation Therapeutic System and Remote Patient Monitoring Device

The present disclosure relates to a smoking cessation therapeutic system and remote patient monitoring device. The smoking cessation therapeutic system is configured to communicate with a plurality of remote patient monitoring devices to deliver computer-executable instructions to the remote patient monitoring devices to provide digital therapeutics to patients of the patient monitoring devices to facilitate smoking cessation among the patients.

In some embodiments, the smoking cessation therapeutic system utilizes brain-based interactive mechanisms across a number of sensory modalities to facilitate smoking cessation, reduce cravings and prevent relapse. User's cravings are addressed through combinative, guided, cognitive therapies, including a virtual cigarette designed to re-create sensorimotor pleasures associated with smoking, deep breathing exercises that reduce cravings and automated targeted messages educating users about the quitting process and reinforcing successful quitting strategies. Additionally, detailed user data gathered by the application reinforces the benefits of behavioral change—including temporal, monetary, health and wellness improvements.

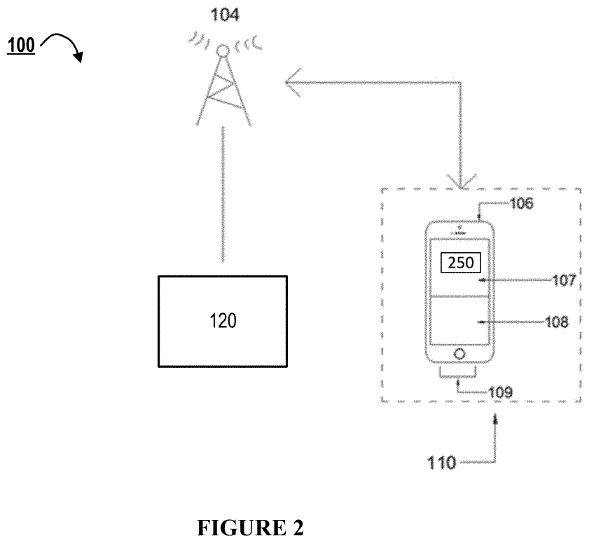

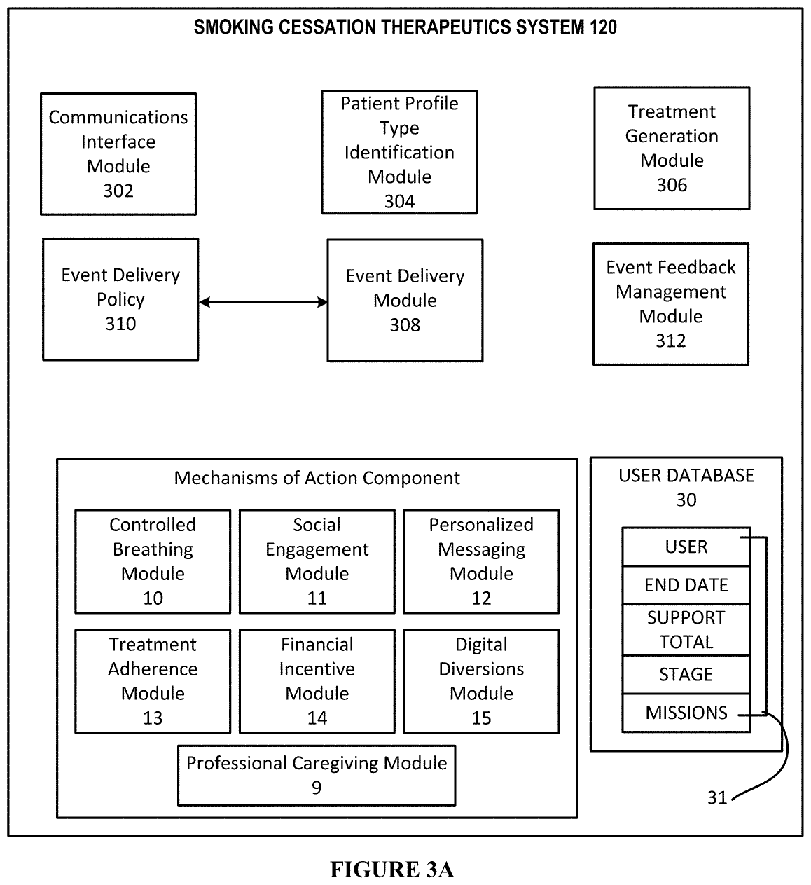

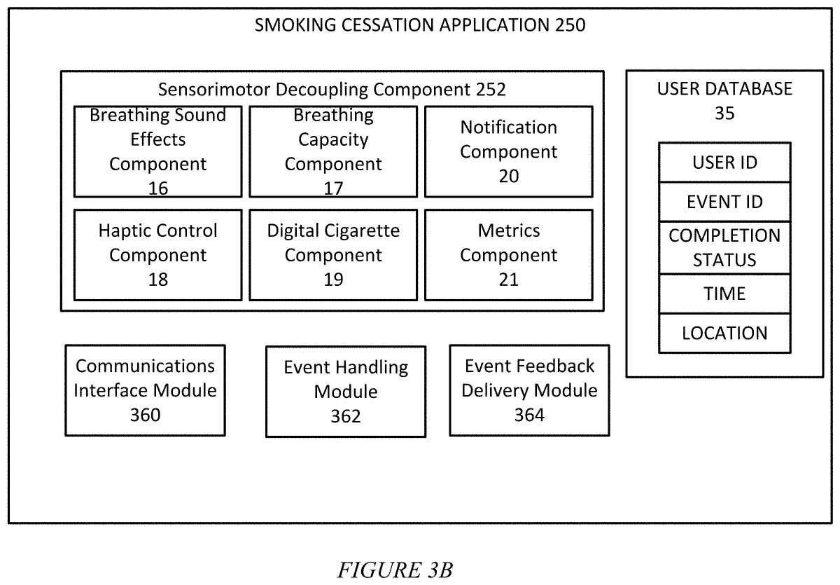

Referring now to FIG. 2, FIG. 2 shows one embodiment of a computer-based smoking cessation therapeutic environment including a smoking cessation therapeutics system and a patient monitoring device communicatively coupled to the smoking cessation therapeutics system via a network. The smoking cessation therapeutic system operates in a computer-networked environment and can include a smoking cessation therapeutics system 120, as will be described with respect to FIG. 3A and a plurality of patient monitoring devices 110 that execute a smoking cessation application stored thereon, as will be described with respect to FIG. 3B. The smoking cessation therapeutic system 100 can be similar to the environment shown in FIG. 1A, where a plurality of servers 1106A-N communicate with a plurality of client devices 1102A-N via one or more computer networks 1104. In some implementations, the smoking cessation therapeutics system 120 can be or execute on one or more of the servers 1106A-N, while the patient monitoring devices can be the client devices 1102A.

The patient monitoring device 110 of the system 100 can include client handheld or computer mediums. For example, the patient monitoring device 110 can be a mobile device, a tablet device, a wearable, a laptop, a desktop computer, or a special purpose medical device. The patient monitoring device 110 and the smoking cessation therapeutics system 120 can each include corresponding data store. The patient monitoring device 110 can communicate with the smoking cessation therapeutics system 120 via the network 104 to receive and transmit messages, location information, patient queries, and patient medical records. The patient monitoring device 110 and the smoking cessation therapeutics system 120 (and other components of the system 100) can communicate using HTTP packets that include requests, transaction information, updates, and other forms of data. The communication can occur over any appropriate electronic communication medium or network using any suitable communications protocols—for example, a mobile network.

FIG. 3A is a block diagram of one embodiment of the smoking cessation therapeutics system shown in FIG. 2. As shown in FIG. 3A, the smoking cessation therapeutics system 120 includes modules that include computer-executable instructions that when executed, cause the smoking cessation therapeutics system 120 to perform various functions. Although not shown in FIG. 3A, the smoking cessation therapeutics system 120 includes hardware components that are configured to store and execute the computer-executable instructions included in the modules. In addition, the smoking cessation therapeutics system 120 can include one or more data storage components, such as memory, that can store one or more data structures.

As shown in FIG. 3A, the smoking cessation therapeutics system 120 includes a plurality of modules, components or other entities that are executed by one or more processors of the smoking cessation therapeutics system 120. The components of the smoking cessation therapeutics system 120 can include applications, programs, libraries, scripts, services, processes, tasks and/or any type and form of executable instructions executing on a computing device.

In brief overview, the smoking cessation therapeutics system 120 is configured to execute on one or more computing devices, such as the computing device 1100 shown in FIG. 1C. The smoking cessation therapeutics system 120 can be configured to cause the computing device to perform one or more functions and may be configured to cause various components of the computing device, such as network interfaces, to establish connections with one or more patient monitoring devices and transmit and receive data over connections. The smoking cessation therapeutics system 120 includes a plurality of modules configured to communicate with instances of a smoking cessation application 250 executing on one or more remote patient monitoring devices.

The communications interface module 302 of the smoking cessation therapeutics system 120 is configured to establish communications between the smoking cessation applications 250 executing on the patient monitoring devices and the smoking cessation therapeutics system 120. The communications interface module 302 can generate data packets, such as HTTP data packets, that are transmitted to the network and then routed to a specific patient monitoring device. The communications interface module 302 can be similar to the network interface 1118 described above in relation to FIG. 1C. The communications interface module 302 is configured to interface with a network via a connection such as a LAN or WAN link. For example, the communications interface module 302 can include a network adapter, network interface card, PCMCIA network card, EXPRESSCARD network card, card bus network adapter, wireless network adapter, USB network adapter, modem or any other device suitable for interfacing with a network. The communications interface module 302 is configured to transmit, to the patient monitoring devices, event messages, generated by the one or more components of the smoking cessation therapeutics system. The communications interface module 302 can also be configured to receive response messages sent from the patient monitoring devices that include data about the status of the previously transmitted event messages.

Patient profile type identification module 304 is configured to identify a profile type of a patient. The patient profile type identification module 304 can include computer executable instructions that, when executed, cause one or more processors to identify the profile type of the patient. For example, the patient profile type identification module 304 can be a programming object that includes data and computer executable instructions. The patient profile type identification module 304 can identify a profile type of the patient in response to data received from the patient monitoring device. For example, the smoking cessation therapeutics system can transmit queries to the patient monitoring device, or in some implementations, the patient monitoring device can generate queries from data stored in the smoking cessation application executing on the patient monitoring device. The queries are presented to a patient of the patient monitoring device. The data from the responses to the queries is transmitted to the smoking cessation therapeutics system 120 and analyzed by the patient profile type identification module 304. The queries can be displayed to a patient of the patient monitoring device in the form of a questionnaire. The questionnaire can include general health questions, family health questions, behavioral questions, goals the patient may have, perceived and challenges to achieving those goals. In some implementations, the queries are transmitted to secondary applications via an application program interface (API) or other interface. For example, the patient monitoring device may submit queries to the FitBit API (made available by FITBIT of San Francisco, Calif.) about the patient's relative activity level. The FitBit API can return a data response that the patient profile type identification module 304 incorporates into the analysis for determining the patient profile type. The patient profile type identification module 304 can perform a cluster analysis or other machine learning analysis on the data received in response to the queries to determine a patient profile type of the patient associated with the patient monitoring device.

The patient profile type identification module 304 classifies the patient profile into one or more types based on the data contained in the profile. The patient profile type identification module 304 may classify the profile into a profile type because patients associated a first patient profile type may respond more favorably to a first type of treatment while patients associated with a second patient profile type may respond more favorably to a second type of treatment. In some implementations, the patient profile types can include a motivational type and an executive type. Patients associated with an executive type profile may respond more favorably when allowed to formulate goals and strategies, make decisions, and self-regulate. Patients associated with a motivational type profile may respond more favorably when rewarded for obtaining a predetermined goal.

The treatment generation module 306 is configured to generate smoking cessation treatment plans for patients. The treatment generation module 306 can include computer executable instructions that, when executed, cause one or more processors to generate the treatment plan for the patient. For example, the treatment generation module 306 can be a programming object that includes data and computer executable instructions. The treatment generation module 306 generates the treatment plan by generating a collection of messages (also referred to as events). The events are generated by the treatment generation module 306 responsive to the data returned to the smoking cessation therapeutics system 120 in response to the queries generated by the patient monitoring device, the data in the patient profile, and current stage of the patient along the quit journey, and the patient profile type. The treatment generation module 306 can store the collection of messages in a database of the smoking cessation therapeutics system 120 until each of the messages are transmitted to the patient monitoring device. In some implementations, the treatment generation module 306 can transmit the collection of messages or events to the patient monitoring device along with an event delivery schedule indicating when to present each of the events. In this way, even if the communications interface module of the smoking cessation therapeutics system cannot establish communications with the patient monitoring device at the time an event is to be provided for display at the patient monitoring device, the patient monitoring device is able to display the event without requiring a connection with the smoking cessation therapeutics system. This implementation addresses the technical challenge of requiring connections between the smoking cessation therapeutics system and the patient monitoring device during times when events are to be presented on the patient monitoring device by no longer requiring such connections.

The events generated for a patient are retrieved from the database and transmitted to the patient monitoring device by the event delivery module 308. The event delivery module 308 can include computer executable instructions that, when executed, cause one or more processors to identify the profile type of the patient. For example, the event delivery module 308 can be a programming object that includes data and computer executable instructions. The event delivery module 308 can deliver the events to the patient monitor by transmitting the events to the patient monitor via the communications interface module 302.

The event delivery module 308 can transmit the events to the patient monitoring device according to a schedule. The schedule can be defined by an event delivery policy 310. The treatment generation module 306 can customize the policy to each of the patients based on the profile type, past behavior of the patient, times when the patient is known to be active, and the patient's profile. In some implementations, the policy can include delivery parameters according to which the event delivery module 308 can determine which type of message or event to deliver and the format in which to deliver the message. One of the delivery parameters can be location information received from the patient monitoring device. For example, when the patient monitoring device passes through a geofence, the patient monitoring device may transmit a message to the smoking cessation therapeutics system 120. The policy 310 can indicate which event should be sent to the patient monitoring device in response to the patient monitoring device entering the geofenced location. The policy can also incorporate scheduling information. For example, the event delivery policy 310 can indicate that a first set of events should be delivered to the patient monitoring device on week days and a second set of events should be delivered to the patient monitoring device on weekend days. The event delivery policy 310 can also incorporate other scheduling information, such as a calendaring information. For example, the smoking cessation therapeutics system 120 may access a calendar associated with the patient. The event delivery policy 310 may include instruction to deliver specific events before, during, or after scheduled events.

The event delivery policy 310 can be updated based on the data responses to the events received from the patient monitoring device. For example, the event responses can include data that indicates the status of an event—for example, the event can be displayed and completed, displayed and not completed, displayed and not started, or not displayed. The event delivery policy 310 can be modified such that events are transmitted to the patient monitoring device to increase the number of events that are displayed and then completed. For example, the event delivery policy 310 can be modified to instruct the event delivery module 308 to send a greater relative number of events during times where previously the smoking cessation therapeutics system 120 received responses indicating the event was displayed and completed. For example, the smoking cessation therapeutics system 120 may determine that the patient is more likely to positively respond to events if they are delivered between the hours of 4 PM and 6 PM than compared to the hours of 8 AM and 10 AM.

The event feedback management module 312 receives feedback from the mobile smoking cessation application executing on the patient monitoring device. The event feedback management module 312 can include computer executable instructions that, when executed, cause one or more processors to receive the feedback. For example, the event feedback management module 312 can be a programming object that includes data and computer executable instructions. When generated, each of the events can be associated with a unique identifier. Each event record can also include the status of the event, such as, but not limited to undelivered, delivered, displayed, completed, and incomplete. After the display of an event by the mobile smoking cessation application, the mobile smoking cessation application can transmit a status message regarding the displayed event. The mobile smoking cessation application can transmit the status message when any change is made to the status of an event. For example, the mobile smoking cessation application can send a new status message when the event is displayed, started, and completed. In other implementations, the mobile smoking cessation application can send update status messages at predetermined intervals responsive to a status update policy. For example, the mobile smoking cessation application can send a status message every hour that includes data about the current status of each event.

When the event feedback management module 312 receives data identifying the status of an event from the mobile smoking cessation application, the event feedback management module 312 can update the record of the event in the user database 30. The event status message can include the original, unique event identifier sent with the event. The event feedback management module 312 can use the event identifier to retrieve the event record from the user database 30 and update the status of the event. In some implementations, the event feedback management module 312 can then set a bit to act as a flag to the treatment generation module 306. The flag to the treatment generation module 306 can indicate the treatment generation module 306 should reprocess the event records to determine if the treatment plan should be modified.

In other implementations, the treatment generation module 306 can periodically (e.g., once an hour or once a day) scan the user database 30 for updates to the event records. In some implementations, the treatment generation module may be configured to generate subsequent events to include in the treatment plan of the patient monitoring device based on the data responses received from the patient monitoring device. The treatment generation module may analyze the data responses received and update the event delivery policy of the patient monitoring device based on the data responses. This feedback mechanism enables the smoking cessation therapeutics system to dynamically update the treatment plan for the patient of the patient monitoring device, resulting in an adaptive, personalized treatment plan for the patient.

The smoking cessation therapeutics system 120 also includes a user database 30. The user database can include records for a plurality of patients that are updated based on interactions between the patient, via a corresponding patient monitoring device, and the smoking cessation therapeutics system 120. Each user record 31 in the user database 30 can include information about the patient, such as, an end date by which time the patient would like to implement the behavioral change; a support total that can indicate the total amount of financial incentives the patient's supporters have pledged to date; a current stage in the behavioral modification processes; and actions—e.g., missions, habits, strategies, preventative strategies, etc.—that can be or have been performed by the user to help divert the user's attention to emergencies. Each patient's (or user's) record can be a table that includes an entry for each of the events associated with the patient. The entries can include the status of the event, the event's identifier, and the mechanism of action of each event.

The smoking cessation therapeutics system 120 also includes one or more mechanism of action modules, such as a controlled breathing module 10, a social engagement module 11, a personalized messaging module 12, a treatment adherence module 13, a financial incentive module 14, and a digital diversions module 15, and a professional caregiving module 9. Additional details relating to the different mechanism of action modules will be provided below.

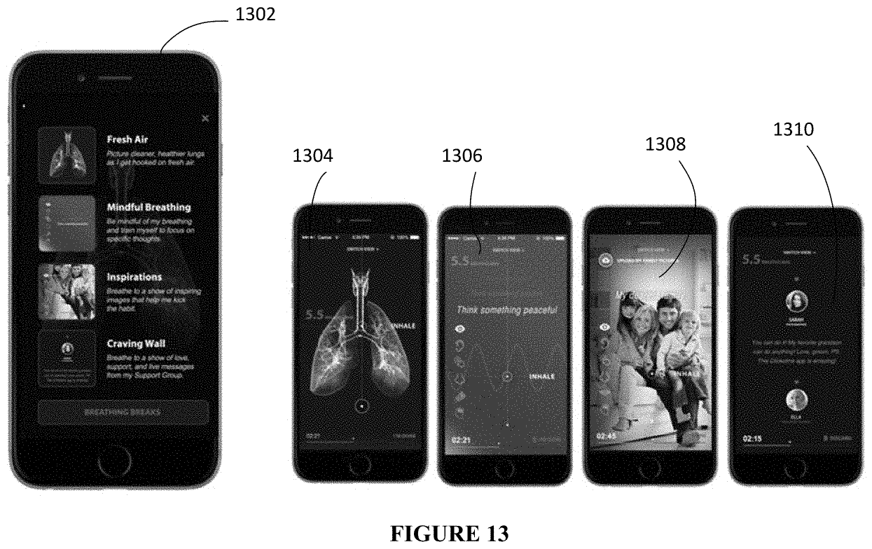

The controlled breathing module 10 includes computer executable instructions that, when executed, cause a processor to generate a controlled breathing event. When displaying the controlled breathing event, the patient monitoring device displays a notification for the patient to begin a controlled breathing exercise. The event also includes instructions for the patient monitoring device to display haptic feedback indicators. For example, a pulsing meter that the patient uses to pace his breath. The controlled breathing module 10 is described further in relation to FIG. 13.

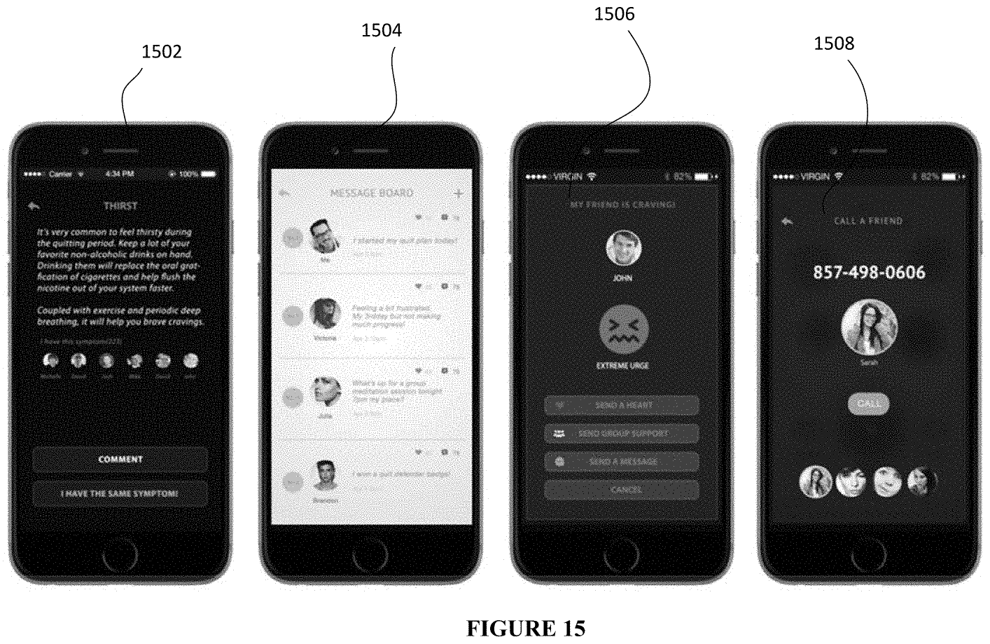

The social engagement module 11 includes computer executable instructions that, when executed, cause a processor to generate a social engagement events that bring about social engagement. The social engagement events can be notifications that are generated by the social engagement module 11. The social engagement events can include motivational messages from the patient's community (e.g., friends and family). The social engagement module 11 can store the event in the database 30 for delivery at a time as indicated by the event delivery policy 310. The social engagement module 11 is described further in relation to FIG. 15.

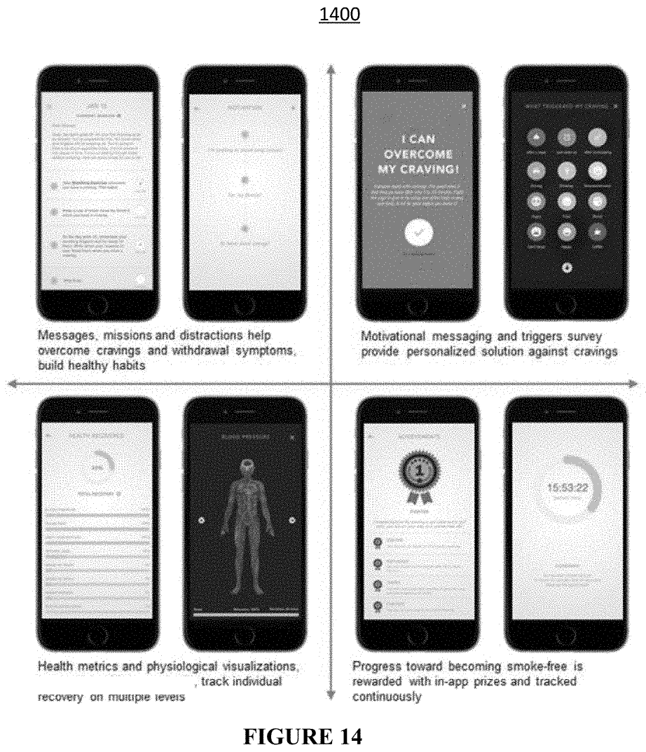

The personalized messaging module 12 includes computer executable instructions that, when executed, case a processor to generate a message event. The message event can include motivational messages that the patient may find motivational. The message events generated by the personalized messaging module 12 can differ from the messaging events generated by the social engagement module 11 in that the personalized messaging module 12 are not generated by the patient's community. The personalized messaging module 12 may generate the messaging events responsive to data stored in the database about events that the patient may find motivating. For example, the messaging event can include a notification to display on the patient monitoring device that indicates how many hours of life the patient will gain back for every cigarette not smoked. The personalized messaging module 12 is described further in relation to FIG. 14. The personalized messaging module 12 can generate personalized messages by included content of the patient, for instance, photos of the patient and their family, photos extracted or retrieved from one or more social networking websites at which the patient has an account. In some implementations, the messages can include content relating to social networking connections of the patient. In some implementations, the messages can be delivered via services native to the mobile platform (e.g., Apple Push Notification service) or through the telephone network (e.g., short messaging service (SMS)).

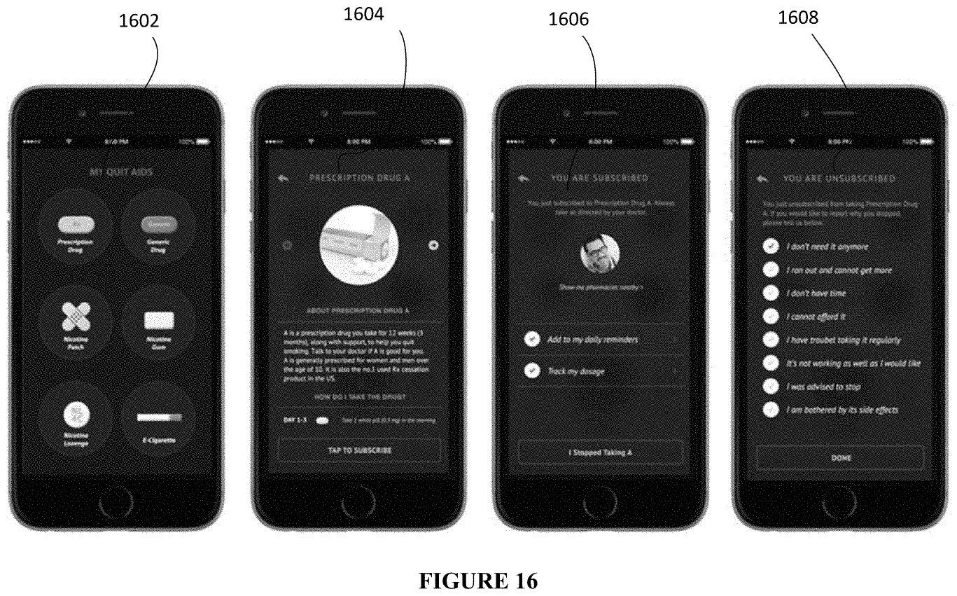

The mechanisms of action component also includes a treatment adherence module 13. The treatment adherence module 13 includes computer executable instructions that cause a processor to generate an adherence event. Adherence events are transmitted to and then displayed on the patient monitoring device to help the patient adhere to treatment regiments. For example, the adherence event can cause the patient monitoring device to display a reminder to the patient to take a specified medication. The treatment adherence module 13 is described further in relation to FIG. 16.

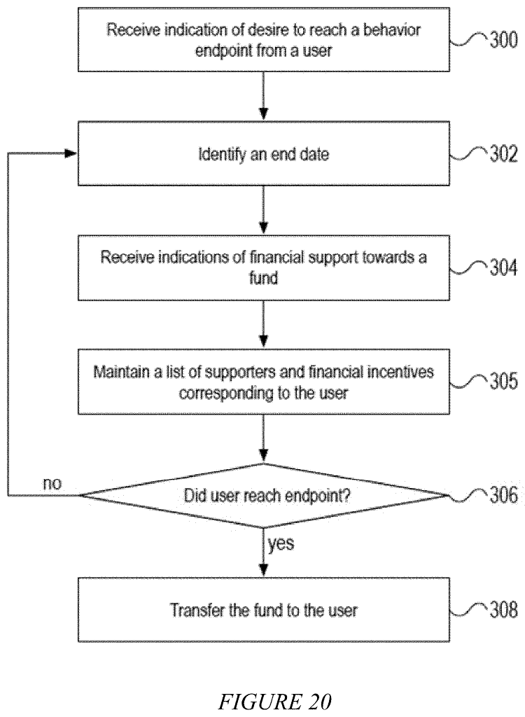

The financial incentives module 14 includes computer executable instructions that cause a processor to generate a financial incentive event. As described further in relation to FIG. 20, financial incentive events can help the patient form new habits. The financial inventive events provide notifications of financial incentives the patient can obtain if the patient performs a predetermined activity. For example, a financial incentive may be provided to the patient for every day the patient does not smoke a cigarette.

The mechanisms of action component also includes a digital diversions module 15. The digital diversions module 15 includes computer executable instructions that cause a processor to generate digital diversions event. As described further below, the digital diversions event is transmitted to the patient monitoring device and causes the patient monitoring device to display a digital diversion of the patient. In some implementations, the digital diversion event can include a notification message configured to distract the patient from a craving. The digital diversion event can include a notification message that, when displayed on the patient monitoring device, prompts the patient to play a video game—one example of a digital diversion.

The mechanisms of action component also includes a professional caregiving module 9. The professional caregiving module 9 includes computer executable instructions that cause a processor to generate a professional caregiving event. In some implementations, the professional caregiving event includes a message from a professional caregiver. The events generated by the professional caregiving module 9 can be similar to those generated by the social engagement module 11. The professional caregiving events can differ from the social engagement events in that the professional caregiving event can include a message from a professional caregiver rather than a person in the patient's social circle. The professional caregiving event can cause the patient monitoring device to display an interface through which the patient can interact with a live human professional trained in the art of behavior change. The interaction can be mediated through asynchronous messaging or synchronously through voice and text messages. In some implementations, the treatment generation module of the smoking cessation therapeutics system can generate professional caregiving events when the treatment generation module determines that the patient needs a greater relatively level on intervention.

The smoking cessation application 250 includes a user management module 232, a predictive analysis module 238, a staging module 236 and a user interface module 234. The smoking cessation application 250 also includes a communications interface module 360, an event handing module 362, and an event feedback delivery module 364. The smoking cessation application 250 also includes a sensorimotor decoupling component that includes a breathing sound effects component 16, a breathing capacity component 17, a haptic control component 18, a digital cigarette component 19, a notification component 20, and a metrics component 21.

The communications interface module 360 coordinates the communication between the smoking cessation application 250 and the smoking cessation therapeutics system 120. The communications interface module 360 can encode status messages sent to the smoking cessation therapeutics system 120 and decode events transmitted from the smoking cessation therapeutics system 120. After receiving data packets from the smoking cessation therapeutics system 120, the communications interface module 360 can route the data to the appropriate component of the smoking cessation application 250. For example, the communication interface module 360 can receive event messages and then store the event messages in the user database 35 prior to their display by the patient monitoring device.

The event handing module 362 is configured to display the event on the patient monitoring device. For example, the event handling module 362 can retrieve events form the database 35 or receive events directly from the smoking cessation therapeutics system 120 via the communications interface module 360. The event handling module 362 causes the patient monitoring device to display the message, notification, or other action contained in the event message. The event handling module 362 can also generate interact input field that are displayed to the patient by the patient monitoring device after the event is displayed. The input fields are configured to receive an indication form the patient that the patient completed or engaged with the displayed event. In some implementations, the event handling module 362 can interface with other components of the patient monitoring device. For example, if the event included a notification for the patient to exercise, the event handling module 362 could interface with an accelerometer of the patient monitoring device or a GPS module to determine if the patient performed the required exercises.

Responsive to displaying the event, the event handling module 362 can update the event record in the database. For example, after displaying the event, the event handling module 362 can update the event record to “displayed” and after receiving an indication the event was completed via a displayed input field, the event handling module 362 can update the event record to “completed.”

The event feedback delivery module 364 is configured to monitor the status of the records in the database 35 and generate status messages to be delivered to the smoking cessation therapeutics system 120, via the communications interface module 360, when the event records are updated. In some implementations, the event feedback delivery module 364 can continuously monitor the event records, and, in other implementations, the event feedback delivery module 364 can monitor the event records at predetermined intervals or after notification from the event handling module 362 that one of the event records was updated.

In some implementations, some components of the smoking cessation therapeutics system 120 may operate on one or more centralized or distributed servers connected to remote patient monitoring devices of patients or users while other components of the smoking cessation therapeutics system 120 may operate on the patient monitoring devise remote to the servers.

The components of the smoking cessation therapeutics system 120 are further described below. As an overview, the smoking cessation therapeutics system 120 includes one or more computing devices that are configured to execute computer-readable instructions for executing a smoking cessation application 250 to bring about a behavioral change in users of the smoking cessation therapeutics system. The smoking cessation application 250 is configured to decouple sensorimotor experiences of smoking experienced by a user from non-nicotinic reinforcers through initiation of exercises, missions, and other mechanisms of action. The smoking cessation therapeutics system 120 can include one or more centralized servers configured to communicate with a plurality of computing devices, such as patient monitoring devices or other client devices. In some implementations, some or all of the components of the smoking cessation therapeutics system 120 can execute on the patient monitoring devices, such as client devices. Users seeking to change their behavior can communicate with the smoking cessation therapeutics system via the client devices. These client devices can serve as an input devices configured to receive input from the users. The input can be transmitted to the smoking cessation therapeutics system as network packets that include a payload carrying information of the user. In some implementations, the client devices can communicate with one or more other devices of the user, such as wearables, or other devices that may monitor, sense or otherwise interact with the user.

The smoking cessation therapeutics system 120 can be configured to generate, for each user, one or more messages, via established communication interfaces between the system 120 and the patient monitoring device of the user. The messages include computer executable instructions that cause the patient monitoring device to display a notification to the user via the patient monitoring device. As described above, a collection of the messages may form a treatment plan. Each of the messages can include an identifier that enables the messages, the patient monitor's response thereto, to be tracked and updated in the database of the smoking cessation therapeutics system 120.

The therapies and treatments may be delivered to the patient monitoring device of the user in the form of messages that that are transmitted to and then displayed by the patient monitoring device. The messages may include instructions for the user to perform particular missions or exercises. These therapies and treatments can link the digital world to the physical world. For instance, a therapy or treatment can include a breathing exercise. The smoking cessation therapeutics system can provide a user interface on the client device of the user inviting the user to perform a breathing exercise. In some implementations, the smoking cessation therapeutics system may be able to detect whether the user is performing the breathing exercise via one or more wearables attached to the user or via the client device itself. In some implementations, the smoking cessation therapeutics system may be able to detect whether the user has performed the breathing exercise via the patient monitoring device of the user. In some implementations, the smoking cessation therapeutics system may not be able to physically detect whether the user has performed the breathing exercise but may determine that the user has performed the breathing exercise by receiving an input from the user via the user interface of the client device indicating that the user performed the breathing exercise.

The smoking cessation therapeutics system 120 can provide tailored therapies at a personalized level based on one or more parameters of the user. The smoking cessation therapeutics system 120 is adaptive and dynamic in that the smoking cessation therapeutics system 120 is configured to adapt the treatments and therapies based on data received from the plurality of users associated with the smoking cessation therapeutics system 120. The smoking cessation therapeutics system 120 can be configured to receive input and/or feedback from a plurality of users based on the therapies or treatments provided to the users. For example, the patient monitoring devices associated with each of the users can generate messages that are transmitted to the smoking cessation therapeutics system 120. The messages can include user inputted data or data automatically generated about the user's progress. The feedback from the users can be used by the smoking cessation therapeutics system to modify or adjust therapies based on the feedback. In addition, the smoking cessation therapeutics system is configured to work with prescription drugs and configured to develop and provide therapies and treatments to users to taper users' reliance on other forms of intervention. In addition, by bringing about behavioral change, the smoking cessation therapeutics system can reduce or completely eliminate the need for the user to take prescription medicines, thereby improving the health of the user and as a result, reducing the associated costs of healthcare of the user.

In some implementations, the patient monitoring device is a digital handheld device with a wireless connection to the internet such as iPads, Kindles, smart phones, wearable device, and other similar touch screen devices. Such devices may be easily carried by a user and used for smoking cessation anywhere. In some implementations, the hand held device can include wearable devices. For example, the devices can be smart watches, such as the Apple watch or Android watches and the wearable devices can include Fitbits or other fitness trackers. In some implementations, the smart watches, wearable devices, or other hand held devices, include biofeedback sensors. The biofeedback sensors can include sensors that monitor heart rate, temperature, movement, or activity.

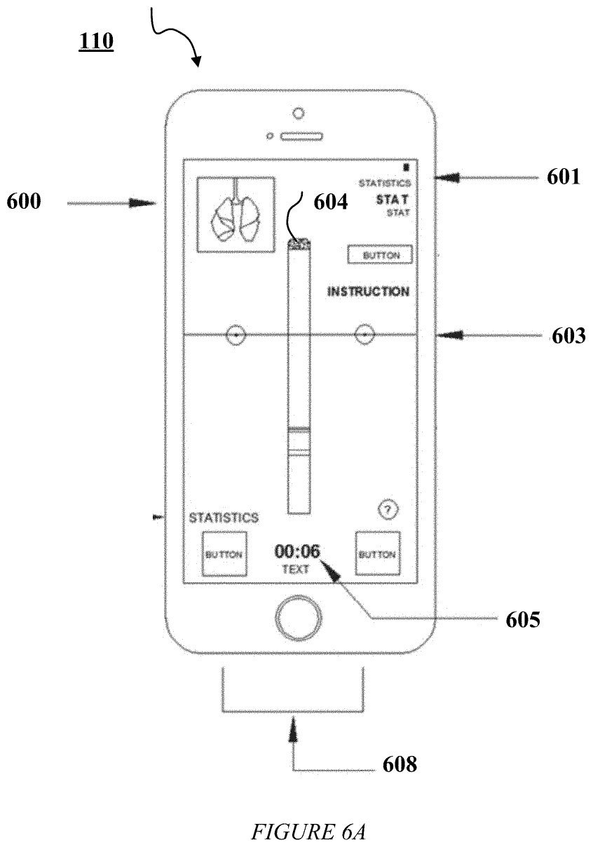

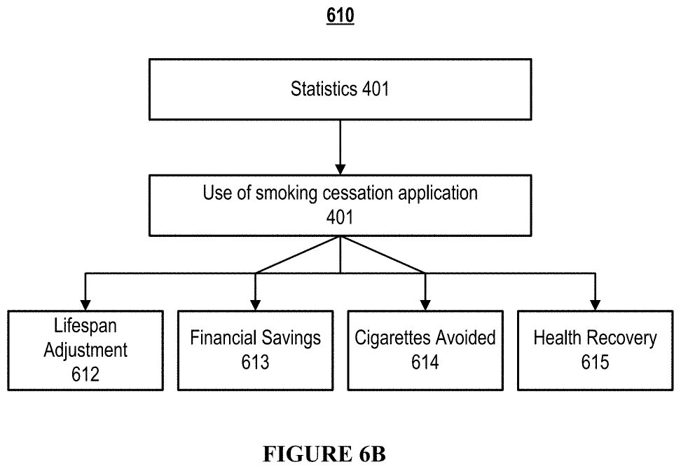

As described further below in relation to FIG. 6A, the smoking cessation application 250 can include a breathing sound effects component 16. In some implementations, the breathing sound effects component 16 is an application component that executes on the patient monitoring device and generates signals supplied to a speaker on the patient monitoring device. The signals cause the speakers to generate an audio output of one or more breathing sound effects that correspond to a controlled breathing pace. In some implementations, the breathing sound effects component 16 generates the signals that cause the speakers to generate the audio output during controlled breathing exercises. In some implementations, the breathing sound effects component 16 is executed by a remote server of the smoking cessation therapeutics system 120 and generates instructions that are transmitted to the patient monitoring device, which then cause the patient monitoring device to generate the breathing sound effects. In some implementations, the breathing sound effects can include an inhaling and an exhaling sound effect.

As described further below in relation to FIG. 6A, the smoking cessation application 250 can include a digital cigarette component 19. The digital cigarette component 19 can be an application that causes the smoking cessation application 250 to generate and display a digital cigarette. The digital cigarette can extend along a length of the display of the patient monitoring device. The digital cigarette can include a top portion with a lit portion and a bottom portion that includes a cigarette butt.

As described further below in relation to FIG. 6A, the smoking cessation application 250 can include a haptic control component 18. The haptic control component 18 can be an application that provides sensorimotor engagement. The haptic control component 18 can cause the smoking cessation application 250 to cause the patient monitoring device to display a haptic control bar that can extend horizontally across the digital cigarette. The haptic control bar can display as a line along a predefined axis that can extend the length of the digital cigarette. The haptic control bar can train the user to haptically follow the haptic control bar while the haptic control bar slides from the lit portion towards the cigarette butt and from the cigarette butt to the lit portion, such as when the user performs a breathing exercise.

Sensorimotor pleasures of smoking can be coupled with non-nicotine reinforcers to induce neuroplastic change and promote goal-attainment. A conventional cigarette is a powerful system to deliver rapid, self-titrated doses of nicotine to the human brain. However, smoking addiction is also sustained by non-nicotinic mechanisms as well. Sensorimotor experiences (e.g. mouth, hand, lung, visual, olfactory, etc.) provide significant reinforcement when coupled with nicotine surges. In some embodiments, the computer-executable application is configured to repeatedly expose the user to non-nicotinic stimuli (sounds of a cigarette, visual representation of a cigarette, controlled deep breathing) to reinforce sensorimotor pleasures of smoking, mitigate cravings, and increase the likelihood of nicotinic uncoupling. Through this medium, both the executive and motivation systems can be systematically engaged through the multiple interaction modalities described, allowing users to remain calm and to control and systematically reduce and respond to cravings.

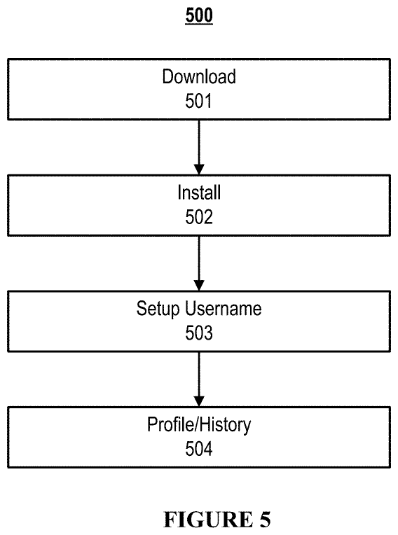

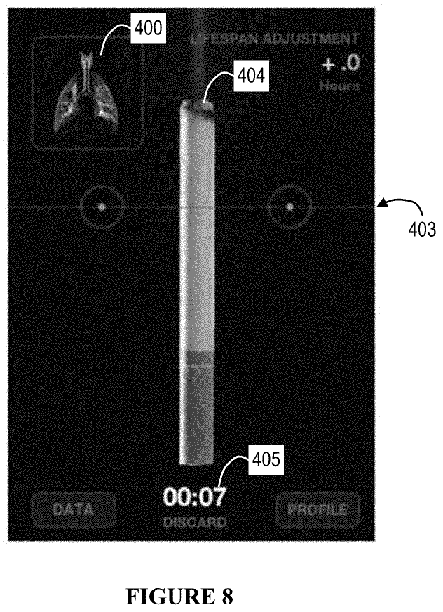

FIG. 5 illustrates a block diagram of a method 200 for installing a computer-executable application associated with the smoking cessation therapeutics system 120 onto a patient monitoring device 110. The method 200 includes downloading the computer-executable application (BLOCK 501). In some implementations, the computer-executable application is downloaded via an app store such as the Apple App Store, Google Play App Store, Amazon App Store, or the Windows App Store. The computer-executable application is then installed on the patient monitoring device 110 (BLOCK 502). In some implementations, the computer-executable application automatically installs onto the patient monitoring device 110 after completion of the download step (BLOCK 501). In other implementations, a user of the patient monitoring device 110 must install the computer-executable application via an install application after the download of the computer-executable application is complete. Upon first startup of the computer-executable application on the patient monitoring device 110, the user of the patient monitoring device 110 is prompted to create a user name and security password (BLOCK 503). The user of the patient monitoring device 110 can also create a user profile (BLOCK 504). The user profile can be created from the user of the patient monitoring device 110 or for a patient associated with the patient monitoring device 110. Generating a user profile is further described in relation to FIG. 8.

In some implementations, once the computer-executable application is installed on the patient monitoring device 110, messages can be sent from the smoking cessation therapeutics system 120 to the patient monitoring device 110 via communication interfaces of the patient monitoring device 10 and the smoking cessation therapeutics system 120. In some implementations, the messages can be delivered to the patient monitoring device 110 and displayed on the patient monitoring device 110 by system notifications sent from the smoking cessation therapeutics system 120. For example, the messages can be loaded into and displayed in a system tray of the patient monitoring device 110 (e.g., the notification dropdown of an iOS device). A user can interact with the notifications by touching user interface elements, such as buttons or icons, displayed in the message notification on a display of the patient monitoring device, reading the contents of the message, and by providing feedback on a message's efficacy and usefulness. The messages can be delivered to the patient monitoring device 110 over the network 104. In some implementations, the messages are delivered at time intervals specified by the user (e.g., the user selects to receive the messages at 2:200 PM), at times specified by the smoking cessation therapeutics system 120, at times selected to correlate with anticipated craving windows. In some implementations, the messages are contextualized messages that are linked to the user's cravings and withdrawals. For example, when the smoking cessation therapeutics system 120 predicts the user is about to experience a craving, the smoking cessation therapeutics system 120 can transmit a message to the patient monitoring device 110, for display to the user of the patient monitoring device 110, with a thought provoking question or an inspirational statement of why the user should quit smoking. An example question can include “David, stop and think about why you're quitting: for my children.”

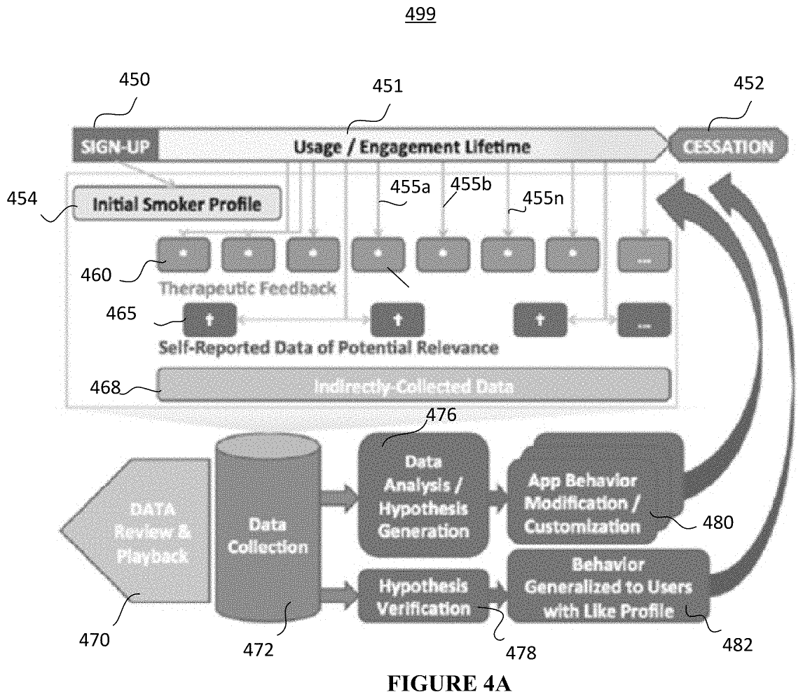

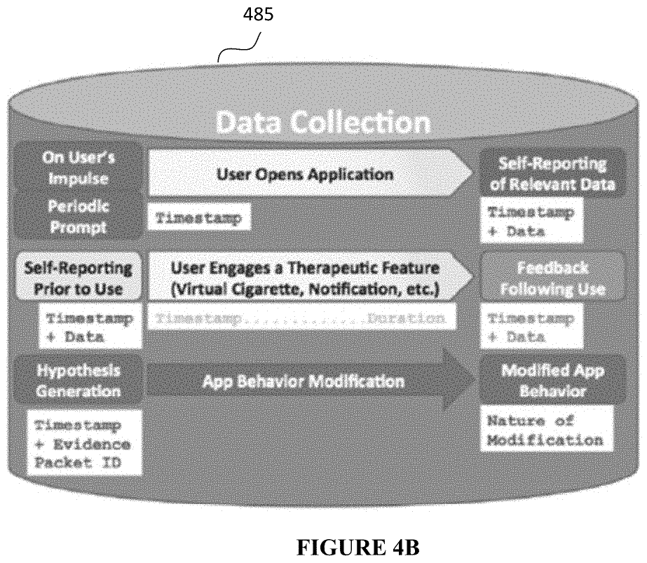

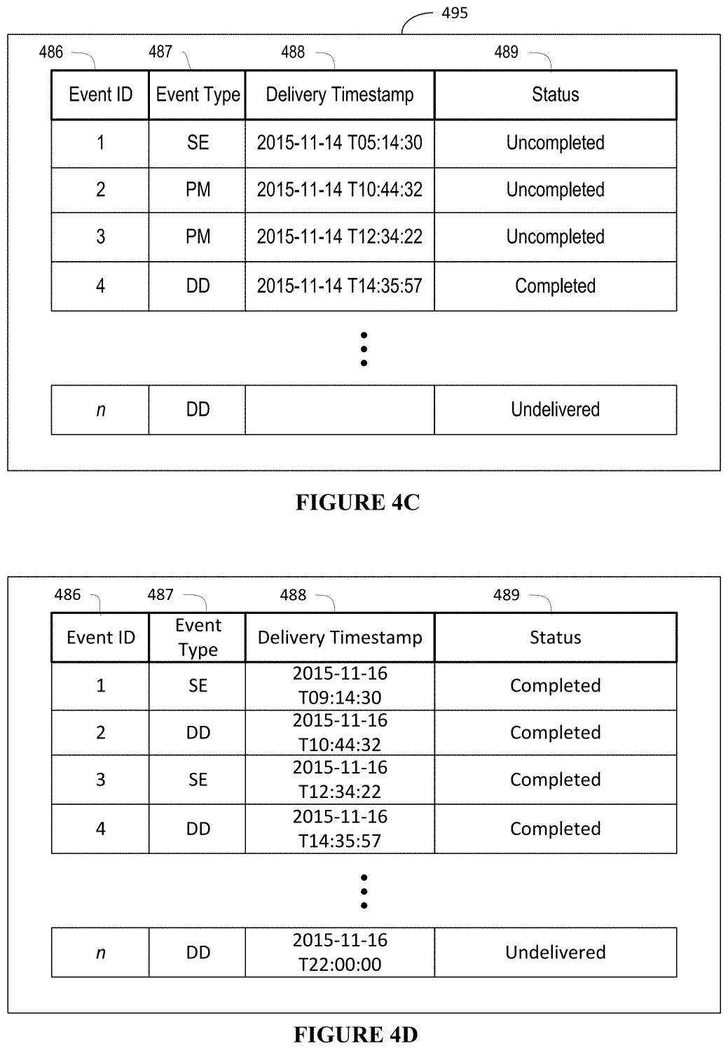

FIGS. 4A and 4B illustrate block diagrams of data flow between the application and back end servers monitoring usage data, location information, and other data useful to analyzing user behavior and customizing the application's therapeutic offerings and interface. The system can include client handheld or computer mediums, networking systems, and back-end networking systems. The client system (e.g., the patient monitoring device 110) and back-end system can each have a corresponding data store such as local storage medium, location data storage medium, and back-end system data storage. The patient monitoring device 110 can communicate with location networking system to receive messages, location information, etc. Additionally, the back-end system can transmit data to and receive data (e.g. de-nicotinized cigarette usage information, location information, profile information, application interaction data, etc.) received from the patient monitoring device 110. The components of the system can communicate using HTTP packets including data requests, transaction information, updates, etc. Communication can occur over any appropriate electronic communication medium or network using any suitable communications protocols. One skilled in the art would appreciate that these are presented merely as an example of a data flow between various types of online systems. However, the present disclosure is intended to encompass data flows between a local client and a variety of other online systems including back end data servers.

In some embodiments, the system delivers contextualized interactions to the patient monitoring device 110 based on the user profile, user statistics, and preference. In some implementations, the contextual interactions are selected using an actionable heuristic model of cognitive-behavioral change guiding the delivery of smoking cessation content through two types of cognitive feedback: executive and motivational. Executive control can be empowered when users formulate goals and strategies, make decisions, and effectively self-regulate. An ascending pathway corresponding to motivation is activated when users initiate and maintain desired behaviors, experience pleasure, and receive rewards.

The data flow 499 illustrated in FIG. 4A begins with the patient signup period 450, continues through the engagement period 451, until the cessation period 452. During the patient signup period 450, the patient monitoring device displays a plurality of input fields associated with a patient enrollment questionnaire. The response entered into the plurality of input fields are transmitted to the smoking cessation therapeutics system and stored in a user database as an initial smoker profile 454. The profile 454 is a record in the database and can be stored as a table that includes a unique identifier for the patient, medical history data, behavior history data, and other data described herein.

As illustrated, the profile 454 is updated throughout the engagement period 451. The profile 454 can be updated through a plurality of mechanisms such as therapeutic feedback 460, self-reported data 465, and indirectly collected data 468. As described above in relation to FIG. 3A, the smoking cessation therapeutics system 120 can generate event messages that are transmitted to the patient monitoring device according to an event delivery policy. The events can be transmitted to and then displayed by the patient monitoring device as a notification, digital diversion, or other mechanism of action. The event handling module 362 can monitor the status of the event, such as determining when the event is completed or if the event is not completed by the patient. The event feedback delivery module of the smoking cessation application can transmit a status message of event back to the smoking cessation therapeutics system 120 as therapeutic feedback at time points 455a-455n. The event handling module 362 can also provide an input field where the patient can enter self-reported data. For example, the patient can self-report non-compliance with dosing of a medication or the lapse of a bad habit, such as smoking. The self-reported data can be collected by the event-handling module and then transmitted back to the smoking cessation therapeutics system 120 via the event delivery module at time points 465.

In some implementations, the patient monitoring device can interface with other devices, such as wearables. From the wearables or other devices, the patient monitoring device can collect additional data, such as activity data. The data collected via the other devices can be transmitted back to the smoking cessation therapeutics system 120 and input into the profile 454. In some implementations, the indirectly collected data 468 can also include data that is transmitted to the smoking cessation therapeutics system 120 via the patient's user community. For example, if a friend of the user witnesses the patient smoking, the friend can report the patient to the smoking cessation therapeutics system 120.

Along the engagement period 451, as the data is collected 472, the data entered into the profile 454 is reviewed 470. Review of the data can follow two general tracks. The first analysis track is used to update the treatment plan of the patient as more data is provided to the smoking cessation therapeutics system 120. The second analysis track modifies the internal heuristics the patient profile time identification module uses to classify the profile 454 of future users. Additionally, the second analysis track can modify the internal heuristics used by the treatment generation module in developing treatment plans for the current and future user.

In the first analysis track, the collected data is analyzed (BLOCK 476) to generate a hypothesis about which mechanisms of actions may work better for the patient. The hypothesis can be generated responsive to the therapeutic feedback 460 received at each of the time points 455. For example, if the status messages received at the time points 455 associated with social engagement events indicate the event was uncompleted, but the status messages received in response to controlled breathing indicate the event was completed, the smoking cessation therapeutics system 120 may form a hypothesis that the patient responds better to controlled breathing events. The first analysis track can then continue to update the patient's treatment plan (BLOCK 480) to include more controlled breathing events.

During the second analysis track, the data flow can include hypothesis verification (BLOCK 478). During this step, the smoking cessation therapeutics system 120 can determine if each of the mechanisms of action work are effect for the patient profile type. At BLOCK 482, the mechanisms of action that are more effective can be used to generate more events for future users of the systems described herein.

FIG. 4C illustrates a table 495 that stores event records. The table 495 can be stored in, for example, a database such as the above-described user database. The table 495 can include a plurality of event records. Each event record can be added to the table 495 as a new row. Inso, the table 495 can be stored on a patient monitoring device to which the events are delivered by the smoking cessation therapeutic system. Each event record can include a first entry column 486 for storing the event's identifier, a second entry column 487 for storing the event type, a third entry column 488 for storing a delivery timestamp of the event, and a fourth entry column 489 for storing a status of the event. The table can also include additional entry columns for each record. For example, if the event is a personalized message event or social engagement event, the record can also include an entry of a message to be displayed to a patient via the display of a patient monitoring device.

As further described above, the treatment generation module 306 can periodically scan the table 495 in the user database 30 for updates to the event records of the table 495. Based on the change in status to the event records, the treatment generation module 306 can update the event delivery policy 310 of the patient monitoring device and the event types that are part of the patient's treatment plan based on the status updates. This feedback mechanism enables the smoking cessation therapeutics system 120 to dynamically update the treatment plan for the patient of the patient monitoring device, resulting in an adaptive, personalized treatment plan for the patient.

As one example, the table 495 includes a plurality of event records (1-n) as they are initially generated and stored by the treatment generation module 306. The table 495 indicates that event #1 is a social engagement (SE) event that was delivered to the patient monitoring device for display at 5:14 AM on Nov. 14, 2015. The event record indicates that the event was not completed by the patient. The events #2 and #3 were personalized messages and were also not completed. The event #4 was a digital diversion (DD) event and was completed by the patient. The event n can be set for a delivery in the future, and the status is set to undelivered. The event #4 can be delivered to the patient monitor by the event delivery module at a time prescribed by the event delivery policy.

The smoking cessation therapeutic system 120 can determine the status of each event via the event feedback management module 312, which can receive a response from the patient monitoring device. The response can include the event identifier of the event transmitted to the patient monitoring device. When the patient monitoring device receives the event or upon display of the event on the patient monitoring device, the smoking cessation application on the patient monitoring device can identify any actions the patient performs on the patient monitoring device. Based on the actions performed, for instance, interacting with the user interface of a breathing exercise (if the event was for a breathing exercise), the patient monitoring device, via the smoking cessation application, will transmit a response to the smoking cessation therapeutics system 120 indicating that the patient completed or did not complete the breathing exercise.

FIG. 4D illustrates the table 495 on Nov. 16, 20215 (e.g., two days later than the table 495 illustrated in FIG. 4C). As the event records are updated, the treatment generation module can analyze which of the events were successfully completed—for example, by determining which of the events have a status of “completed” as compared to “incomplete” or “not started.” As illustrated, the treatment generation module 306 modified the delivery time of the event #1 and modified the event type of the event #2 and #3. For example, after receiving status event messages each day that the event #1 was not completed, the treatment generation module 306 may be able to determine that the patient was not yet active (for example, asleep) and altered the event delivery policy such that the first event was delivered later in the day. The treatment generation module 306 may also run an analysis to determine which of the delivered event types are most likely to have a “completed” status. For example, after a number of days, the treatment generation module 306 can analyze the past events to determine that the private message events had a completed status only 15% of the time and the digital diversion events had a complete 80% completed status. Accordingly, in the table 495 of FIG. 4D, the treatment generation module 306 added additional digital diversion events and removed private message events.

FIG. 6A illustrates a block diagram of a screen shot of one example implementation of the computer-executable application executing on the patient monitoring device 110. The computer-executable application can include computer-executable instructions, which when executed by the patient monitoring device 110, can cause the patient monitoring device 110 to provide, for display on a display of the patient monitoring device 110, a representation of the de-nicotinized cigarette 604 (also referred to as a digital cigarette 604) with a haptic input guided deep breathing control bar 603, breath capacity representation 600, and timer 605. The display of the de-nicotinized cigarette 604 is generated by the computer-executable application using the breathing sound effects component 16, the breathing capacity component 17, the haptic control component 18, and the digital cigarette component 19. The computer-executable application executing on the patient monitoring device 110 can generate breathing sound effects from a speaker 608 for guidance, display targeted notifications, and real-time metrics 601 that estimate life-years preserved and money saved. The targeted notifications can be provided for display by a notification component 20 of the computer-executable application and can generate and deliver messages at predetermined intervals. The real-time metrics 601 can be generated by the metrics component 21 of the computer-executable application.

The digital cigarette component 19 can be configured to cause the patient monitoring device to generate and display the digital cigarette 604. In addition, the digital cigarette component 19 can be configured to provide a user interface through which the user can interact with the digital cigarette 604. For example, the digital cigarette 604 is ignited by the user via a touch screen interface 107 and the sound and visualization of a lit cigarette are displayed on the touch screen interface 107. When lit, the end of the de-nicotinized cigarette 604 flames to the sound of a striking match, with the flame slowing dying down to a slow burn, as happens with a traditional cigarette. The haptic control component 18 generates the haptic control bar 603 that raises and lowers to the sound of deep breathing, leading the user through a controlled deep breathing exercise. The haptic control bar line 603 moves lower as the user inhales and the breath capacity representation 600, in one embodiment a graphic of human lungs, change color as a representation of air moving into or out of the user's lungs. The change in color of the graphic of the human lungs can correlate to the position of the haptic control bar 603 relative to the cigarette butt portion of the lit portion of the de-nicotinized cigarette. When exhaling, the haptic control bar 603 moves upward signaling exhaling and the color disappears. In some implementations, the mindful breathing application presents the user with a sinusoidal curve that scrolls across the screen. The breathing sound effects component 16 can generate sounds that correspond to inhalation and exhalation sounds. The user can be instructed to mindfully breath in and out with the scrolling of the curve. For example, the user can breathe in as the curve peaks and the break out as the curve bottoms out. The metering of the breathing rate can be controlled by the user or the system. Haptic interactions with the moving line itself encourage sensorimotor engagement and provide motor measures of user adherence. In one embodiment, the haptic control bar 603 can be self-guided (e.g., controlled by the user via touch screen) or computer controlled. For example, the haptic control bar 603 can slide between the lit portion and the cigarette butt at a speed corresponding to the breathing pace. In some implementations, the haptic control bar 603 slides from the lit portion towards the cigarette butt at the same time the inhaling sound effect is output by the breathing sound effects component 16 and the haptic control bar 603 slides from the cigarette butt to the lit portion at the same time the exhaling sound effect is output by the breathing sound effects component 16.

The smoking cessation application 250 can include a timer component that generates the timer 605 at the bottom of the representation informs a user as to the total time spent during the deep breathing exercise. During the exercise, the user is able to see the statistics 601 concerning his or her usage of application. In some implementations, multisensory stimuli can be played during the mindful breathing exercise. The multisensory stimuli can include mindful narration or inspirational images that play during the breathing exercise.

In some implementations, the application can also present to the user a craving rating indicator. The indicator can initially be set automatically by the predictive analysis module 238. For example, if the predictive analysis module 238 predicts the user is about to experience a craving, the craving indicator can be set to a relatively high level. As the user performs the mindful breathing exercise, the craving indicator can lower. If the user does not feel the craving passing, the user can reset the craving indicator to a relatively higher level, which will cause the mindful breathing exercise to continue for a longer period of time.

FIG. 6B illustrates a block diagram 610 for computing the above-discussed statistics 401. The statistics 401 are created automatically based upon application usage 611, which enables the application to determine a lifespan adjustment 612, the total number of cigarettes avoided 614, financial savings to the user 613, and a health recovery 615.

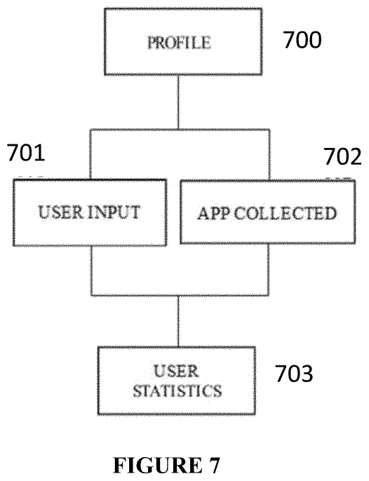

FIG. 7 illustrates a block diagram of the data input into a profile 700. At a first level, the profile 700 includes user inputted data 701 and application collected data 702. The profile can also include user statistics 703, such as those discussed above that can include cost savings achieved (both in the short term and over a life span), extension of life, and the number of de-nicotinized cigarettes utilized. In some implementations, functional aspects of a user's engagement with the smoking cessation therapeutic system will be stored on the server 100 and are available for review and playback by the user and/or a health professional. In some embodiments, the data can be displayed on the display 107 of the patient monitoring device 110. This can include data collected before and after the user engages with one of the smoking cessation therapeutic system's various therapeutic features, for example, the virtual cigarette 404, deep breathing controls 403, system notification 2000, or any combination thereof. Time-varying effects are modeled statistically within person coupling and uncoupling of behavioral state and application usage, actual cigarette smoking, cravings, stress levels. In one embodiment, it includes the totality of times when the smoking cessation therapeutic system initiates a customization over the baseline behavior for the user, based on data collected from him or her to date.

FIG. 8 illustrates a screenshot of one example implementation of the smoking cessation application 250 executing on the patient monitoring device 110 similar to the block diagram described above in relation to FIG. 7. The screenshot illustrates a de-nicotinized cigarette with haptic input guided deep breathing control bar, breath capacity representation and timer. The de-nicotinized cigarette is ignited by the user upon a touch screen interface and the sound and visualization of a lit cigarette manifest within the medium. When lit, the end of the cigarette flames to the sound of a striking match, with the flame slowing dying down to a slow burn, as happens with a traditional cigarette. The haptic control bar raises and lowers to the sound of deep breathing, leading the user through a controlled deep breathing exercise. The line moves lower as the user inhales and the breath capacity representation (a graphic of human lungs) change color as a representation of air moving into or out of the users' lungs. When exhaling, the haptic control bar moves upward signaling exhaling and the color disappears. Haptic interactions with the moving line itself encourage sensorimotor engagement and provide motor measures of user adherence. The control bar can be self-guided (e.g., controlled by the user via touch screen) or computer controlled. A timer at the bottom of the representation informs a user as to the total time spent during the deep breathing exercise. FIG. 6A illustrates another embodiment of the de-nicotinized digital cigarette.

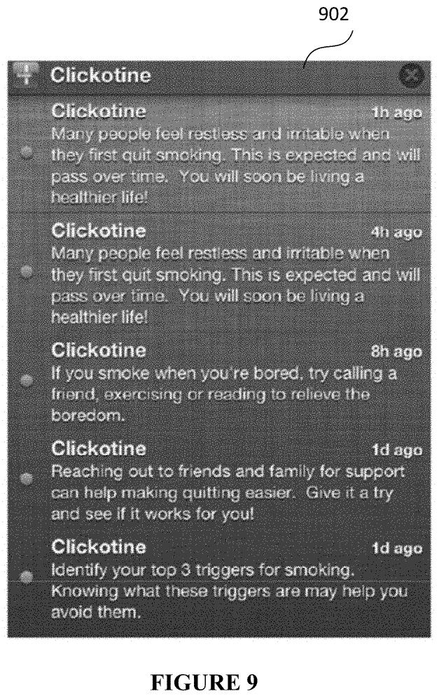

FIG. 9 is a screenshot 902 illustrating messages delivered to the application. Messages are sent to the application via SMS/MMS or any other similar messaging technology (such as MMS, e-mail, etc.) and other forms of communication readily available. The messages are delivered at time intervals specified by the user, or by default, to correlate with anticipated craving windows. In some implementations, the messages are delivered to the application as push notifications from a push notification server communicating with the computer-executable application installed on the patient monitoring device.

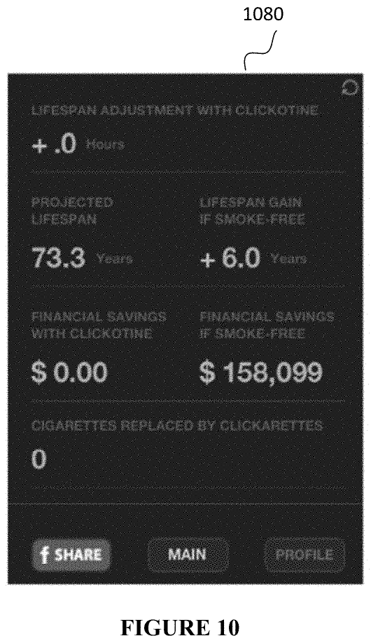

FIG. 10 is a screenshot 1080 illustrating a user profile that is maintained within the application illustrating figures essential to smoking cessation reinforcement such as cost savings achieved, extension of life, number of de-nicotinized cigarettes utilized, or any combination thereof. Functional aspects of a user's engagement with the smoking cessation therapeutic system can be stored and available for review and playback by the user and/or a health professional. This can include data collected before and after the user engages with one of the smoking cessation therapeutic system's various therapeutic features, for example, virtual cigarette, deep breathing, messaging, system notification, etc. It can also include data solicited from the user automatically, impulsively and periodically relating to aspects of his or her experience, which may bear relevance to his or her journey towards full cessation, for example, real cigarette usage, cravings, and stress levels. In addition, it includes an accumulation of modifications made when the smoking cessation therapeutic system initiates a customization over the baseline behavior for the user based on data collected from him or her to date.

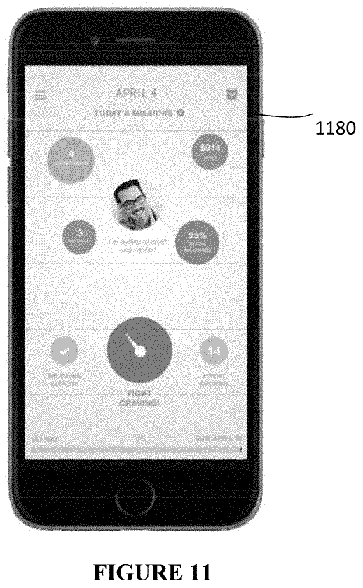

FIG. 11 is graphical representation of a patient monitoring device interface 1180 illustrating a central dashboard that enables a user to access various functionalities/therapeutic features and keep track of quit journey. In some implementations, the user can personalize with photo of choice, including a self-portrait or that of loved ones.

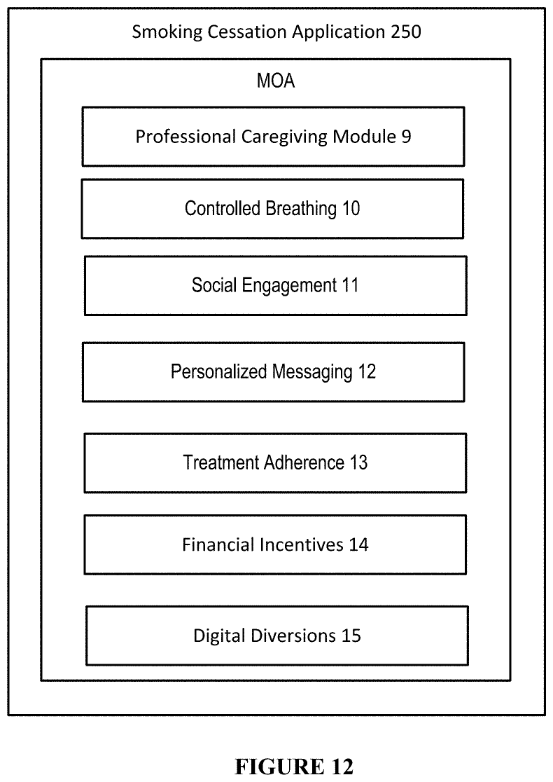

FIG. 12 illustrates seven different mechanisms of action the smoking cessation application is configured to provide. The different mechanisms of action include any of the mechanisms of action described herein, such as controlled breathing, personalized messaging, social engagement, treatment adherence, financial incentives, and digital diversions, and professional caregiving. The controlled breathing includes mindful breathing experience coupled with multisensory stimuli. The personalized messaging includes adaptive text messages tailored to user profile. The social engagement includes real-life support from friends, family and the community. The treatment adherence includes an individualized daily reminder system for prescription and over-the-counter smoke cessation therapies. The financial incentives mechanism includes a tracking system that unlocks monetary rewards when a user successfully abstains from the undesired habit and repeats desired habits. The digital division mechanism of action distracts the user for, by example, providing a video game for the user during craving episodes and other emergencies. The professional caregiving mechanism determines when targeted intervention by a human professional trained in the art of behavior change may be necessary, and connects the patient directly with the caregiver.

FIG. 13 illustrates a patient monitoring device interface for controlled breathing including graphical representations relating to fresh air, mindful breathing, inspirations and social connections that serve as a craving wall. During the breathing exercise, the application can provide an indication of carefully metered breaths and provide a multi-sensory experience, including mindful narration, for example, asking the user to think of something peaceful, among others. Images of loved ones can be displayed to encourage the user to think of their health and the importance of being healthy for their loved ones.

FIG. 14 illustrates graphical representations 1400 of a patient monitoring device interface for personalized messaging as well as for displaying physiological visualizations and progress towards becoming smoke-free via prizes and rewards. The messages, missions and distractions that can be provided via the smoking cessation application 250 and system described herein can help overcome cravings and withdrawal symptoms and help build healthy habits. The messages can be motivating. In some implementations, surveys can be provided to the user via the user interface to provide a personalized solution to the craving or withdrawal symptom based on the responses to the surveys. The user can also be motivated by earning badges and awards via the smoking cessation application 250. The badges and awards can be earned based on good habits and performance; for example, if the user does not smoke for a week, the user can receive a badge corresponding to the achievement.

FIG. 15 illustrates graphical representations 1502-1508 of a patient monitoring device interface for social engagement. Smoking behavior spreads through social ties, while interconnected people can stop smoking in tandem. Behavior change can be achieved within “quit communities” through real-time support, cooperative quit planning & goal setting, and mutual motivation. Via the smoking cessation application 250, a user can communicate with loved ones and other users that are trying to quit smoking to help resist cravings.

In some implementations, the patient records stored in the database can also include entries for relatives, friends, and other contacts in the patient's community. The records can include the social media handles for the community contacts. For example, the records can include the Twitter handle of the community contacts. The social engagement module 11 can scan Twitter for mentions of the patient in the feeds of the community contacts. The mentions collected by the social engagement module 11 can form the basis for the messages presented to the patient during social engagement events.

In some implementations, the social engagement module can create automatic social groups called quit communities. The social engagement module can create the groups based on criteria, such as geographic location, stage in the quit journey, profile types, demographics, neurobehavioral profiles, genetic profiles, or similar interests.

FIG. 16 illustrates includes graphical representations 1602-1608 of a patient monitoring device interface for treatment adherence options. In some implementations, the smoking cessation application 250 can improve effectiveness of pharmaceutical treatments and over-the-counter quit aids by helping users adhere and by tracking their usage. The application can help a user navigate complex dosing schedules, provide reminders to take medications, track when medications are taken and identify improvements in the user's journey to quit based on the reduced number of cravings the user has, among others.

In some implementations, the smoking cessation application 250 can track financial rewards unlocked when a user abstains from undesirable behaviors and performs desirable behaviors. The financial incentives can be included in missions to change behavior. The rewards can be formulated as refunds or reimbursements from institutional partners responsible for or interested in the care of the user. For example, an insurance company may provide a user with a monthly refund if the user can show, through the completion of missions that the user has stopped smoking or taken up a healthy activity. Other institutional partners may include advertisers, healthy lifestyle service providers, or other entities that may have an interest or desire in helping the user quit smoking. For instance, a gym may offer a user who has successfully completed one or more missions discounts at the gym, either in the form of reduced gym payments, additional services provided by the gym (for instance, free personal training), among others. In addition, another person that is part of the user's support circle or someone interested in having the user quit smoking may create missions or financial incentives to motivate the user to quit smoking. For instance, a mother may use the smoking cessation therapeutic system to generate one or more personalized missions and associate a financial incentive. For instance, the mother may provide the smoking cessation therapeutic system instructions authorizing the smoking cessation therapeutic system to charge her $50 if her son completes one or more missions. These missions may be selected by the mother or recommended by the smoking cessation therapeutic system itself. The son, upon completion of the missions, may be awarded the $50 via the smoking cessation therapeutic system.

In some implementations, the smoking cessation application 250 can provide digital diversions to help users engage in new behaviors or to stop behaviors. The digital diversions can be timed and targeted diversions to the user to help the user cope with cravings, withdrawal symptoms, and relapses. The digital diversions can help divert the user's attention from the cravings and withdrawal symptoms so the user does not perform the bad habit. The digital diversions can include presenting a game to the user or diversions with real-world components, such as taking a walk.

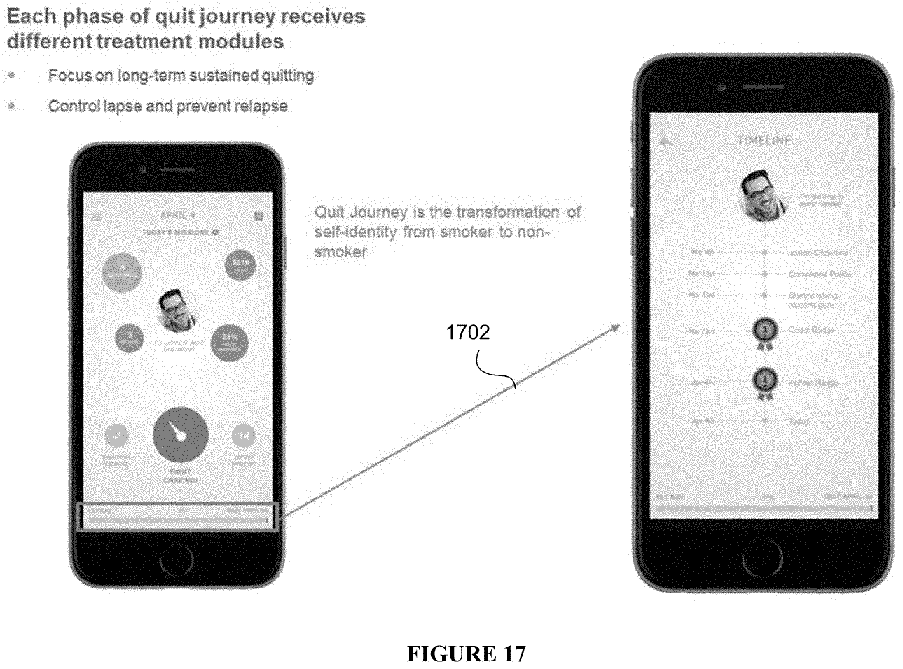

FIG. 17 illustrates graphical representations of a patient monitoring device interface for displaying various phases of the journey to quitting smoking. The smoking cessation application 250 can determine which phase of the quit journey the user is on based on the performance of the user. The smoking cessation application 250 can determine the phase based in part on the frequency of cravings, the ability to resist the cravings, among others. The smoking cessation application 250 focuses on long term sustained quitting and can help control lapses and prevent relapses. The quit journey is the transformation of the user identifying themselves as a smoker to a non-smoker. The user interface shown in FIG. 11 can display a timeline of achievements of the user.

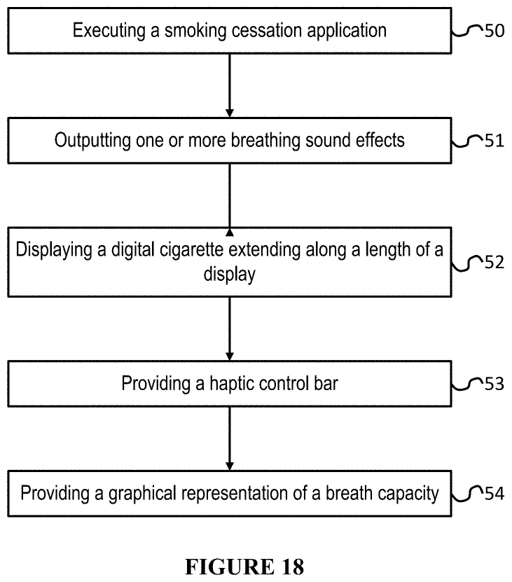

FIG. 18 illustrates a block diagram of a method for decoupling sensorimotor experiences of smoking from reinforcers. The method includes executing a smoking cessation application (BLOCK 50). The method also includes outputting one or more breathing sound effects (BLOCK 51). The method also includes displaying a digital cigarette extending along a length of a display (BLOCK 52). The method can include providing a haptic control bar (BLOCK 53), and providing a graphical representation of a breath capacity (BLOCK 54).

The method includes executing a smoking cessation application (BLOCK 50). The application can be the smoking cessation application 250 described above. Components of the smoking cessation application can be executed locally on a patient monitoring device, remotely on a networked server, or on a combination thereof. The patient monitoring device and the networked server can include one or more processors that execute instructions that cause the processor to perform the methods described herein. In some implementations, the smoking cessation application is configured to decouple sensorimotor experiences of smoking from non-nicotinic reinforcers.

The method can also include outputting one or more breathing sound effects (BLOCK 51). The breathing sound effects can be generated by the above described breathing sound effects component 16 of the smoking cessation application 250. The breathing sound effects can simulate the inhalation and exhalation sounds. The inhalation and exhalation sounds can be generated by the breathing sound effects component 16 at a predetermined breathing pace during a breathing exercise.

The method can include displaying a digital cigarette (BLOCK 52). The digital cigarette can be similar to the digital cigarette described above in relation to FIG. 6A. The digital cigarette is generated by the smoking cessation application for display on a patient monitoring device, such as a mobile phone. The digital cigarette mimics the appearance and functions of a real cigarette. For example, the digital cigarette can extend along the length of the patient monitoring device's display. The digital cigarette can include a top, lit portion and a bottom, cigarette butt portion.

The method can also include providing a haptic control bar (BLOCK 53). As illustrated above in FIG. 6A, the haptic control bar 403 can extend horizontally across the digital cigarette. The haptic control bar can be generated by the haptic control component 18 of the smoking cessation application 250. The haptic control component controls the placement of the haptic control bar along a predefined axis that extends across the displayed digital cigarette. For example, the haptic control component can control the sliding of the haptic control bar from the lit portion of the digital cigarette to the bottom portion of the digital cigarette. As the haptic bar slides towards the bottom portion of the digital cigarette, the lit portion can glow a brighter orange and red color to mimic the appearance of a real cigarette as a user inhales on the real cigarette. At this same time, the breathing sound effects component can generate an inhalation sound effect that is output by the patient monitoring device. Similarly, as the haptic bar slides towards the top portion, the breathing sound effects component can generate an exhalation sound effect that is output by the patient monitoring device. In some implementations, the smoking cessation application generates a message that includes text that instructs the user of the patient monitoring device to follow haptically the haptic control bar as the haptic control bar slides between the upper and bottom portions of the digital cigarette. In some implementations, the smoking cessation application causes the haptic control bar to slide at a speed that corresponds to a breathing pace the user should attempt to mimic. As the user follows the haptic control bar, the user can perform breathing exercises where the user breaths at a pace to match the pace of the inhalation and exhalation sounds generated by the breathing sound effects component. The breathing exercise in combination with moving the haptic control bar and the generation of the breathing sound effects by the breathing sound effects component of the smoking cessation application provides a non-nicotinic stimulus to the user of the smoking cessation application.

The method can also include providing a graphical representation of breath capacity (BLOCK 54). In some implementations, the graphical representation generated by the smoking cessation application is similar to the breath capacity representation 400 described above in relation to FIG. 6A. The smoking cessation application can change the appearance of the generated breath capacity representation as the haptic control bar slides between the upper and lower portions of the digital cigarette. For example, the smoking cessation application can display the breath capacity representation with a first appearance when the haptic control bar is positioned toward the lit portion of the digital cigarette and with a second appearance when the haptic control bar is positioned toward the butt portion of the digital cigarette.

The present disclosure also describes various implementations of a smoking cessation therapeutic system including a social financial incentive mechanism of action. The smoking cessation therapeutic system includes one or more computing devices that are configured to execute computer-readable instructions to bring about a behavioral change in users of the smoking cessation therapeutic system. The smoking cessation therapeutic system can include one or more centralized servers configured to communicate with a plurality of computing devices, such as client devices. Users seeking to change their behavior can communicate with the smoking cessation therapeutic system via the client devices. These client devices can serve an input devices configured to receive input from the users. The input can be transmitted to the smoking cessation therapeutic system as network packets that include a payload carrying information of the user. In some implementations, the client devices can communicate with one or more other devices of the user, such as wearables, or other devices that may monitor, sense or otherwise interact with the user.

The smoking cessation therapeutic system can be configured to provide therapies and treatments to users. The therapies and treatments may include instructions for the user to perform particular missions or exercises that help distract the user from cravings associated with the behavioral change. These therapies and treatments can link the digital world to the physical world. For instance, a therapy or treatment can include a breathing exercise. The smoking cessation therapeutic system can provide a user interface on the client device of the user inviting the user to perform a breathing exercise. In some implementations, the smoking cessation therapeutic system may be able to detect whether the user is performing the breathing exercise via one or more wearables attached to the user or via the client device itself. In some implementations, the smoking cessation therapeutic system may be unable to physically detect whether the user has performed the breathing exercise but may determine that the user has performed the breathing exercise by receiving an input from the user via the user interface of the client device indicating that the user performed the breathing exercise.

The smoking cessation therapeutic system can provide financial incentives to the user. The financial incentives or other rewards can serve as a powerful incentive to help users engage in new behaviors to stop undesirable behaviors. The financial incentives can be provided by partners, such as insurance companies or the financial incentives can be crowdsourced. The crowdsourced (or socialized) financial incentives can be provide by the friends, family, or other supporters of the user. The supporters of the user can pledge to financially reward the user if the user obtains an agreed upon behavioral endpoint (e.g., quitting smoking) by an agreed upon end condition, such as a fixed date in the future, a round of affirmation, a biochemical test, etc. The supporters, knowing and interacting regularly with the user, can also collectively (or individually) affirm that the user achieved the behavioral endpoint through the system before the system provides the financial incentive to the user.

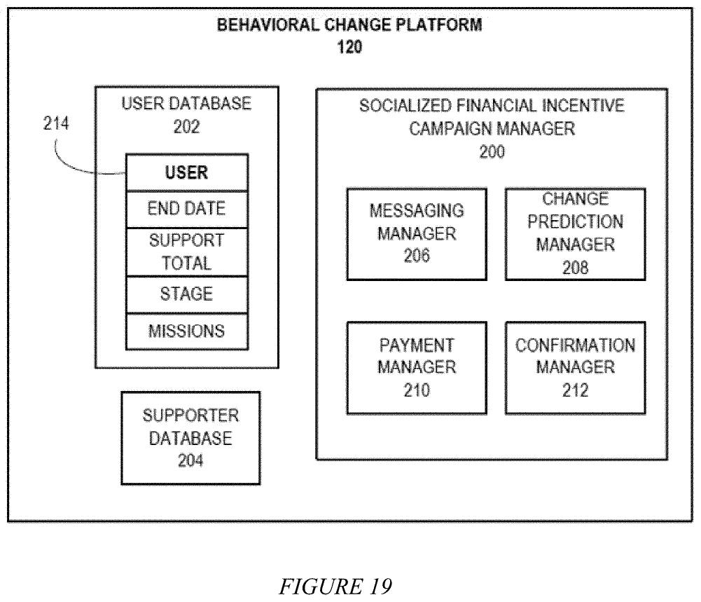

FIG. 19 illustrates a block diagram of a smoking cessation therapeutic system 120. The smoking cessation therapeutic system 120 includes hardware and/or software and is configured to bring about behavioral changes in users of the smoking cessation therapeutic system 120. The smoking cessation therapeutic system 120 includes a plurality of modules, components or other entities that execute within the smoking cessation therapeutic system 120. The components of the smoking cessation therapeutic system 120 can include applications, programs, libraries, scripts, services, processes, tasks and/or any type and form of executable instructions executing on a device, such as a mobile device, computer, or server. In particular, the smoking cessation therapeutic system 120 can include a user database 202 and a supporter database 204. The smoking cessation therapeutic system 120 can also include a socialized financial incentive campaign manager 200 that includes a messaging manager 206, a change prediction manager 208, a payment manager 210, and a confirmation manager 212.

The user database 202 can includes a plurality of tables and entries for each of the users of the smoking cessation therapeutic system 120. In some implementations, the database can include information about each of the users seeking to use the smoking cessation therapeutic system to change one or more behaviors. Examples of behaviors can include smoking, dietary behavior, such as uncontrolled eating, substance abuse, or other dependency-related disorders that have a craving-withdrawal effect. Such behaviors may be habit forming and may have an addiction component to them. In other implementations, the behaviors can include behaviors the user wishes to begin, such as working out, attending classes, or eating a healthier diet. In some implementations, the behaviors can be related to personality traits, for example, schizophrenia, bipolar, and other mood disorders. In some implementations, the behaviors can be related to personal hygiene or other personal habits, such as abnormal sleep patterns. The information about each of the users can be stored in a user entry 214. Each user entry 214 in the user database 202 can include information about the user, such as, an end date by which time the user would like to implement the behavioral change; a support total that can indicate the total amount of financial incentives the user's supporters have pledged to date; a current stage in the behavioral modification processes; and actions—e.g., missions, habits, strategies, preventative strategies, etc.—that can be or have been performed by the user to help divert the user's attention to cravings.

The smoking cessation therapeutic system 120 can also include a supporter database 204. The supporter database 204 can include one or more tables having entries for each of the users listed in the user database 202. Information regarding supporters of a specific user can be stored in one or more tables having entries linked to the specific user. For example, the user may ask for family members and loved ones to support the user in the journey to achieving a predetermined behavioral endpoint. In some implementations, the user's journey toward a behavioral endpoint or attempt to change a habit together with their broadcasting of that journey to supporters of their choosing is referred to as a campaign. The supporters can provide encouragement to the user to help the user reach the behavioral endpoint. In some implementations, the supporters can provide financial incentives to the user if the user reaches the behavioral endpoint. For example, a first supporter of a user may pledge to give the user $100 and a second supporter of the user may pledge to give the user $50 if the user quits smoking.

In some implementations, the supporter can be an entity, such as a company, rather than a person. For example, the supporter may be an insurance company, a physician's practice, an employer, or a gym. The entity can sponsor the user's campaign but rely on individual sponsors to validate whether the user actually achieved the predetermined behavioral endpoint. In this way, the entity does not need to spend resources to independently determine whether the user has achieved the behavioral endpoint, but rather, can leverage the information provided by other vested sponsors to validate that the user did achieve the predetermined behavioral endpoint.

In some implementations, a subset of the supporters may not provide financial incentives and may only provide encouragement to the user or provide confirmation the user reached the endpoint. For example, one supporter of the user may be an insurance company or the user's employer that provides as a financial incentive of a refund or credit on the user's insurance policy if the user reaches an endpoint. In some implementations, a subset of supporters may provide financial incentives and encouragement with pre-specified conditions. For example, the insurance company may require the user have a predetermined number of supporters that are willing to verify whether the user reached the endpoint. The insurance company may agree to lower the user's insurance premium by $100.00 per month if the user stops smoking. The insurance company may require that the user have five supporters verify on the end date that the user reached the endpoint and has in fact quit smoking before the user can receive the reduced premium.

The smoking cessation therapeutic system 120 can include a payment manager 210. The payment manager 210 can handle the processing of payments and pledges by each of the supporters. For example, a web storefront can be provided to the supporters. The supporter can visit the web storefront using a computing device. The supporter can provide pledge information, via the smoking cessation therapeutic system 120, such as a pledge amount and credit card (or other forms of payment) information. In some implementations, the supporter may first enter a pledge amount and then the payment manager 210 may request the payment information from the supporter when the user reaches the behavioral endpoint. The payment manager 210 can also provide the raised financial incentives to the user when the user reaches the behavioral endpoint. For example, the user can provide the payment manager 210 with account information where the financial incentive can be credited when the user reaches the behavioral endpoint.

The smoking cessation therapeutic system 120 can also include a messaging manager 206 and a confirmation manager 212. The messaging manager 206 can provide an interface, through a mobile application or a webpage, for supporters and users to send messages to one another. For example, the supporters can send messages of encouragement to the user using the messaging manager 206. Users can send messages to their supporters providing them with updates about their progress towards achieving the desired behavioral endpoint.

The confirmation manager 212 can confirm with the supporters if the user successfully reached the behavioral endpoint. The confirmation manager 212 can generate messages that can be sent to the supporters and users via the messaging manager 206. For example, the supporters of the user can verify by positively confirming to a message sent from the messaging manager 206 that the user reached the behavioral endpoint before the user receives the financial incentives. The confirmation manager 212 may send messages to the supporters on regular intervals, or near the date the user indicated as the end date, asking whether the supporter can verify that the user reached the behavioral endpoint or has not yet lapsed. For example, the confirmation manager 212 may ask the plurality of supporters of a user if each of the supporters can verify that the user did reach the desired behavioral endpoint of quitting smoking without lapsing. If one of the supporters answers that he saw the user smoking recently (therefore the user did not reach the behavioral endpoint) the smoking cessation therapeutic system 120 may not provide the financial incentives to the user and the supporters may be refunded (or not debited) the amount they pledged. The supporters can also use the messaging manager 206 to notify the smoking cessation therapeutic system 120 and other supporters and user if they detect the user has lapsed. For example, if a supporter witnesses the user smoking when the end point is to stop smoking, the supporter can send a message to the other supporters and user indicating the user has lapsed and it not entitled to receive the financial incentive.

If the confirmation manager 212 determines the user did not reach the endpoint because the confirmation manager 212 did not receive affirmations from a predetermined number of supporters—e.g., reach the affirmation threshold—indicating that the user failed in reaching the behavior endpoint, the confirmation manager 212 can provide a notification the users to set a new end condition—e.g., a new quit date, a new determination date, or a new campaign. In some implementations, the affirmation threshold of supporters can be 1. In some implementations, the affirmation threshold can be based on the number of total supporters. In some implementations, the affirmation threshold can be selected by the user when setting up the campaign. In some implementations, the affirmation threshold can be identified based on a poll conducted by the smoking cessation therapeutics system 120 among a portion or all of the supporters. In some implementations, when the user fails to achieve the behavior endpoint, the confirmation manager 212 can be configured to send messages to each of the user's supporters asking the supporter to indicate whether they wish to continue supporting the user in the new campaign associated with the new end date or if the supporter would like to withdraw their previous pledge.

In some implementations, the confirmation manager 212 can interface with a wearable or other device associated with the user. For example, the confirmation manager 212 can send or receive data from a fitness tracker or a server associated therewith. The confirmation manager 212 can use the data received from the wearable to determine if the user lapsed or whether the user completed a specific mission. For example, the socialized financial incentive campaign manager 200 may assign the user a plurality of missions to complete along the journey to the endpoint. Some of the missions may include completing a physical task, such as walking for 20 minutes. Rather that asking the user if the user completed the physical task, the confirmation manager 212 can analyze data form the wearable to determine if the user completed the physical task. In some implementations, if the confirmation manager 212 determines the user did not complete a predetermined number of missions, the confirmation manager 212 can send a message to the supporters indicating the user lapsed. In some implementations, a combination of messages received form the supports and data received from a wearable device can be used to determine if the user lapsed.

In some implementations, the confirmation manager 212 can interface with a biochemical assay device—e.g., a carbon monoxide breathalyzer, cotinine tester, a digital scale, etc.—to determine whether the user has achieved their pre-specified end condition. For example, if the user is attempting to quit smoking, the confirmation manager 212 may have the user test their urine with a cotinine tester. Cotinine, found in the blood, urine, and saliva of smokers, can indicate if a user has recently smoked. If the confirmation manager 212 determines, via the cotinine tester, that the user recently smoked, the confirmation manager 212 may set a new quit date or set a new campaign. In some implementations, the confirmation manager 212 may determine if the user is progressing toward their goal. For example, if the user's goal is to lose 20 pounds over six months, the confirmation manager 212 may track whether the user is losing the required 3.3 pounds per month to meet the user's goal.

The smoking cessation therapeutic system 120 can also include a change prediction manager 208. The change prediction manager 208 can predict the likelihood that a user currently attempting to reach a behavioral endpoint has reached the endpoint based on one or more of their activities and interactions within the smoking cessation therapeutic system 120. In some implementations, the prediction made by the change prediction manager 208 can be based on the activity of users that have previously used the smoking cessation therapeutic system 120 to reach behavioral change endpoints. To make a prediction of how successful the user is likely to be in reaching the endpoint, the change prediction manager 208 can take into account information such as how long the user performed the habit the user wants to change, the number of missions attempted, the number of missions completed, the amount of supporters the user has, the frequency the user interacts with the smoking cessation therapeutic system 120, the frequency at which the user receives encouragement from the plurality of supporters, the frequency and success rate of the user's interactions with the smoking cessation therapeutics system 120 to abate immediate cravings, among other types of data that can be tracked by the smoking cessation therapeutic system.

The missions can be mechanisms of action, such as activities, that can help the user deal with cravings or can provide distractions for the user when the user faces a craving. The missions can include games, breathing exercises, quizzes, or tasks to be completed in the real world (e.g., brushing teeth or physical exercise). The change prediction manager 208 can compare the user to previous users to determine a likelihood that the user will be able to reach the endpoint.

By collecting the above information about past and present users of the smoking cessation therapeutic system 120, the change prediction manager 208 can create datasets that include the responses, activities, and other information of users that were able to successfully reach the endpoint. The change prediction manager 208 can also create datasets that include the responses, activities, and other information of users that were not able to successfully reach the endpoint. The information and progress of a current user can be compared against the “successful” and “unsuccessful” datasets to calculate a probability the user will be able reach the endpoint. In some implementations, the change prediction manager 208 can use machine-learning algorithms to determine whether the user should be classified as having a high, medium, or low likelihood of reaching the endpoint. In some implementations, the prediction information may be provided to the supporters of the users before the supporters make a pledge so that the supporters can take the prediction information into account when making their pledge. For example, knowing that a user has a low probability of reaching the endpoint, the supporters may determine to provide a higher financial incentive to the user to provide a greater encouragement to reach the endpoint.

FIG. 20 is a block diagram depicting a method of creating a socialized financial incentive campaign to affect a behavioral change endpoint. The method includes starting a socialized financial incentive campaign by receiving an indication of a desire to reach a behavior endpoint (BLOCK 2000). The method includes identifying an end date at which time a system implementing the method determines if the user has reached the behavioral endpoint (BLOCK 2002). The method also includes receiving, from a plurality of supporters, indications of financial support towards a fund (BLOCK 2004). The method can also include maintaining, by the server, for the user, a list of supporters and a corresponding financial commitment for each of the supporters. At the end date, the method can include receiving an indication, from each of the plurality of supporters, whether the user reached the behavior endpoint (BLOCK 2006). Responsive to a confirmation the user reached the behavioral endpoint, the fund can be transferred to the user (BLOCK 2008). However, responsive to failing to receive an indication from the affirmation threshold number of supporters that the user succeeded in reaching the behavior endpoint, the method includes providing a notification to each of the users asking the users participate in a new socialized financial incentive campaign where the user sets a new end date or to withdraw from the campaign.

As set forth above, and also referring to FIG. 19, the method can include starting a socialized financial incentive campaign by receiving an indication of a desire to reach a behavior endpoint (BLOCK 2000). The indication can be received by the smoking cessation therapeutic system 120 from a user. The user can interact with the smoking cessation therapeutic system 120 through a mobile application, such as the smoking cessation application 250 or a website. For example, the smoking cessation therapeutic system 120 may interface with a mobile application, when the user signs into the application for the first time, the application can ask the user what habit (or endpoint) the user would like to start (e.g., what good habit the user would like to start, such as exercising regularly) or stop (e.g., what bad habit the user would like to stop, such as smoking).

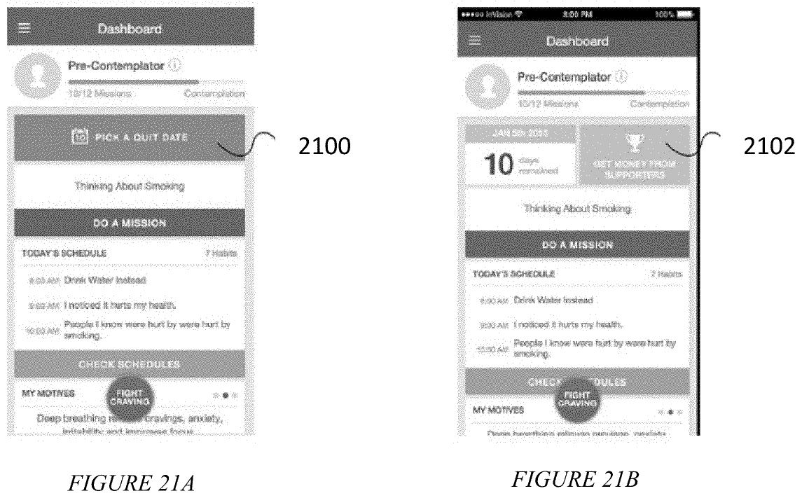

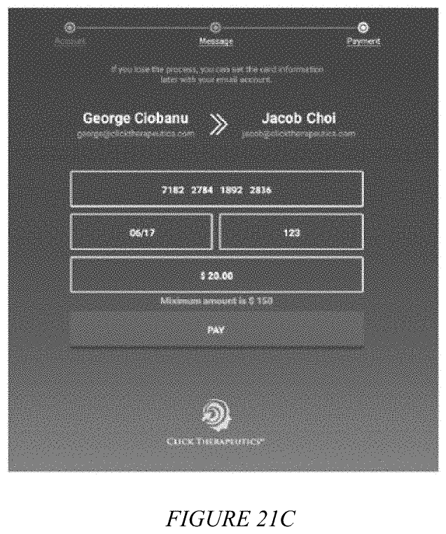

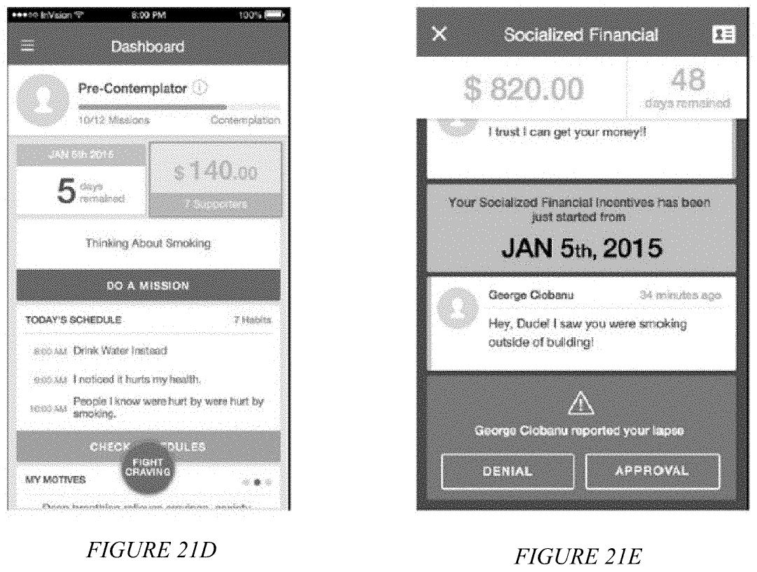

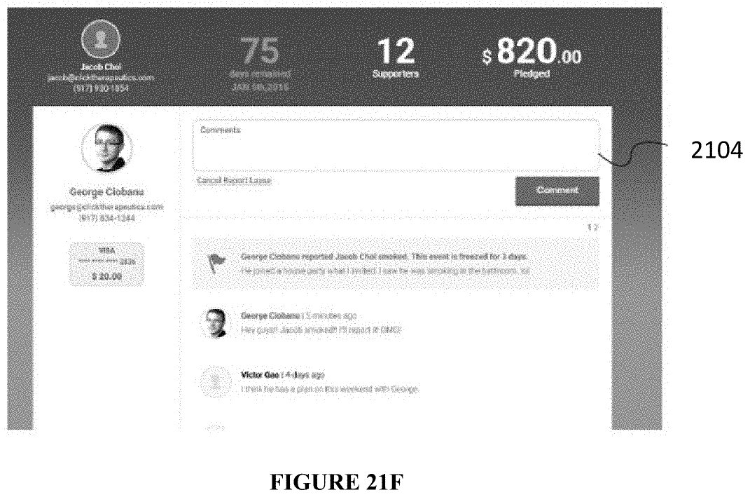

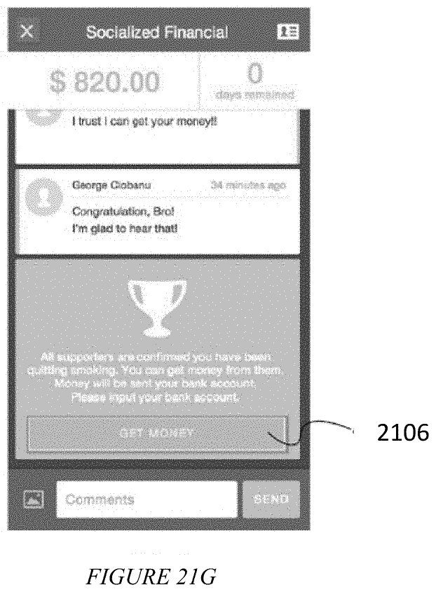

The method can include identifying an end date at which time the smoking cessation therapeutics system 120 can determine if the user achieved the behavioral change endpoint (BLOCK 2002). The end date can indicate by which date the user wanted to start or stop the behavior and achieve the behavior endpoint. In some implementations, the smoking cessation therapeutic system 120 can ask the user by what date the user would like to reach the endpoint. In other implementations, the smoking cessation therapeutic system 120 can ask the user questions, such as how long the user has performed the habit and how difficult the user believes it will be for the user to reach the endpoint, and suggest an end date based on the collected information. The smoking cessation therapeutic system 120 can base the end date on data collected from past users. For example, the smoking cessation therapeutic system 120 may suggest based on information from past users that it takes the average 5-year smoker 4 weeks to quit and the average 10-year smoker 8 weeks to quit. The endpoint, end date, and other user information can be stored in a user entry in the user database 202. FIG. 21A illustrates an example user interface for interfacing with the smoking cessation therapeutic system 120. When the user first uses the application, the application can include a button 2100 that enables the user to select an end date. Once the user selects the end date, the application can calculate the number of days until the end date and provide a countdown to the user. The application can provide an indication of the end date to the smoking cessation therapeutic system 120.

The method can include receiving, from a plurality of supporters, indications of financial support towards a fund (BLOCK 2004). As described above, supporters of the user can provide financial incentives to the user to help incentivize the user to reach the behavior endpoint. Also referring to FIG. 21B, the application can include a button 2102 that enables the user to request financial incentives from supporters. The application may send the requests to the potential supporters using the messaging manager of the smoking cessation therapeutic system 120. The requests may be sent through email, text, or a social media application. In some implementations, the smoking cessation therapeutic system 120 may provide the user with a unique link that the user can post on social media websites or send to potential supporters to enable the supporters to sign up to be a supporter of the user. Clicking on the link can take the potential supporter to a webpage, where the potential support can enter payment information and the amount of money the support is willing to pledge toward the fund that will be given to the user if the user reaches the endpoint. FIG. 21C illustrates an email webpage where a potential supporter can enter pledge information. FIG. 21D illustrates the application, which indicates that the user has seven supporters that have pledged $1210.00 toward the fund.

At the occurrence of the end date, the method can include receiving an indication, from each of the plurality of supporters, whether the user reached the behavior endpoint (BLOCK 2006). For example, the smoking cessation therapeutic system 120 may send a request to each of the plurality of supporters asking if, to the best knowledge of the supporter, the supporter can confirm that the user reached the endpoint. In some implementations, the indication can be received in response to the smoking cessation therapeutic system 120 sending an email, text message, or other message to the supporters asking the supporter to confirm if the user successfully reached the endpoint.

In some implementations, the supporters can send an indication they detected the user lapsing. FIG. 21E illustrates a view of the application when the smoking cessation therapeutic system 120 has received an indication from one of the supporters that the user lapsed. For example, if the user was attempting to quit smoking, the supporter may have witnessed the user smoking. In the user's view of the application, as illustrated in FIG. 21E, the user can confirm or deny the supporter saw the user lapse. In some implementations, the smoking cessation therapeutic system 120 can be configured to enable the support to upload evidence if they witnessed the user lapse. For example, the support may upload an image of the user smoking.

FIG. 21F illustrates an example webpage interface of the smoking cessation therapeutic system 120 a supporter may use to provide an indication to the smoking cessation therapeutic system 120 that the user lapsed. The webpage interface can include a comment box 2104, where the supporter can provide encouraging comments to the user. The comments can be sent, via the smoking cessation therapeutic system's messaging manager to the user. Through the comment box 2104, the supporter can also report a lapse. In some implementations, the report of the lapse or other messages can be sent to the user and also to the other supporters of the user. For example, a lapse notice may be also sent to the other supporters so the other supporters can look for signs the user may have lapsed.

Referring to FIG. 21A and also FIG. 21G, responsive to a confirmation the user reached the behavioral endpoint, the fund can be transferred to the user (BLOCK 2008). In some implementations, each of the user's supporters may have to confirm the user reached the endpoint before the fund is transferred to the user. In other implementations, a majority or predetermined number of supporters may need to confirm the user reached the endpoint before the funds are transferred. FIG. 21G illustrates a view of the user application responsive to the user reaching the endpoint. When the user reaches the endpoint, the application can include an interface 2106 that enables the user to claim the fund. For example, the application may ask the user into what bank account or what debit account the fund should be credited. In some implementations, the user can provide the bank or debit account information when the user establishes an account with the smoking cessation therapeutic system 120, and the funds may automatically be transferred to the user's preferred destination when the supporters confirm the user reached the endpoint. In some implementations, the funds may not be provided to the user, but the user can donate the funds to a charitable organization. The payment manager of the smoking cessation therapeutic system 120 can debit the support's accounts and credit the user's account when the user reaches the endpoint.

In some implementations, if a supporter does not confirm that the user reached the endpoint or if one or more of the supporters report the user lapsed, the smoking cessation therapeutic system 120 can present the user and supporter with a number of options. In one example, in response to a supporter reporting a lapse or not confirming that the user reached the endpoint, the smoking cessation therapeutic system 120 can ask each of the plurality of supporters if the supporter would like to cancel their pledge toward the fund. If a supporter indicates they would not like to cancel their pledge, the smoking cessation therapeutic system 120 can ask the user to set a new end date, and the above method will repeat. In some implementations, if the user does not reach the endpoint, the smoking cessation therapeutic system 120 may ask the user to select a new end date without first asking the supporters if they would like cancel their pledge.

While the invention has been particularly shown and described with reference to specific embodiments, it should be understood by those skilled in the art that various changes in form and detail may be made therein without departing from the spirit and scope of the invention described in this disclosure.

While this specification contains many specific embodiment details, these should not be construed as limitations on the scope of any inventions or of what may be claimed, but rather as descriptions of features specific to particular embodiments of particular inventions. Certain features described in this specification in the context of separate embodiments can also be implemented in combination in a single embodiment. Conversely, various features described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated in a single software product or packaged into multiple software products.

References to “or” may be construed as inclusive so that any terms described using “or” may indicate any of a single, more than one, and all of the described terms.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain embodiments, multitasking and parallel processing may be advantageous.

Having described certain embodiments of the methods and systems, it will now become apparent to one of skill in the art that other embodiments incorporating the concepts of the invention may be used. It should be understood that the systems described above may provide multiple ones of any or each of those components and these components may be provided on either a standalone machine or, in some embodiments, on multiple machines in a distributed system. The systems and methods described above may be implemented as a method, apparatus or article of manufacture using programming and/or engineering techniques to produce software, firmware, hardware, or any combination thereof. In addition, the systems and methods described above may be provided as one or more computer-readable programs embodied on or in one or more articles of manufacture. The term “article of manufacture” as used herein is intended to encompass code or logic accessible from and embedded in one or more computer-readable devices, firmware, programmable logic, memory devices (e.g., EEPROMs, ROMs, PROMs, RAMs, SRAMs, etc.), hardware (e.g., integrated circuit chip, Field Programmable Gate Array (FPGA), Application Specific Integrated Circuit (ASIC), etc.), electronic devices, a computer readable non-volatile storage unit (e.g., CD-ROM, floppy disk, hard disk drive, etc.). The article of manufacture may be accessible from a file server providing access to the computer-readable programs via a network transmission line, wireless transmission media, signals propagating through space, radio waves, infrared signals, etc. The article of manufacture may be a flash memory card or a magnetic tape. The article of manufacture includes hardware logic as well as software or programmable code embedded in a computer readable medium that is executed by a processor. In general, the computer-readable programs may be implemented in any programming language, such as LISP, PERL, C, C++, C#, PROLOG, or in any byte code language such as JAVA. The software programs may be stored on or in one or more articles of manufacture as object code.

What is claimed is:

1. A system, comprising:
at least one server comprising one or more processors and memory having computer-executable instructions stored thereon, the at least one server configured to:
establish a communication channel with a remote device associated with a user;
determine a profile type for the user based on data received from the remote device of the user, the data including query responses;
generate, using the profile type for the user, an event management policy to include a plurality of events to be transmitted to the remote device via the communication channel at a corresponding plurality of times in accordance with a delivery schedule defined by the event management policy, each event of the plurality of events specifying one or more activities to be performed by the user associated with the remote device;
transmit, to the remote device via the communication channel, a first event of the plurality of events of the event management policy in accordance with the delivery schedule to cause an application executing on the remote device to a first action corresponding to the first event and to record response data corresponding to an activity performed by the user in response to the first action via the application;
receive, from the remote device via the communication channel, the response data identifying an event identifier corresponding to the first event, a status identifier indicating the activity performed by the user in response to the first action via the application on the remote device, and a delivery timestamp indicating a time at which the first event is provided;
store, on an activity log for the user maintained in the memory, responsive to the receipt of the response data, a record corresponding to the first event provided to the remote device, the record including the event identifier, the status identifier, and the delivery timestamp;
modify, using the event identifier, the status identifier, and the delivery timestamp of the record stored on the activity log generated from the response data from the remote device, the delivery schedule of the event management policy for the user associated with the remote device; and
transmit, to the remote device via the communication device, a second event from the plurality of events in accordance with the modified delivery schedule to cause the application executing on the remote device to perform a second action corresponding to the second event to induce a change to at least one action performed by the user in response to the second action corresponding to the second event via the remote device.

2. The system of claim 1, wherein the received data identifies a time duration indicating a length of time taken by the user in performing the activity in response to the presentation of the data of the first event via the remote device, and
wherein the at least one server is further configured to store, on the activity log, the record including time duration and to modify at least one of the plurality of events based on the time duration of the record.

3. The system of claim 1, wherein the at least one server is configured to select, from a plurality of event management policies, the event management policy based on the profile type for the user.

4. The system of claim 1, wherein the at least one server is configured to receive, from the remote device, the response data generated by the remote device in response to an interaction with the application on the remote response subsequent to the presentation of the data corresponding to the first event, the first event corresponding to at least a controlled breathing event, a social engagement event, a personalized message event, a treatment adherence event, an incentive event, and a digital diversion event.

5. The system of claim 1, wherein the at least one server is further configured to modify at least one of the times specified by the event delivery schedule using the event identifier, the status identifier, and the delivery timestamp of the record stored on the activity log.

6. The system of claim 1, wherein the at least one server is further configured to transmit the first event of the plurality of events to the remote device, receipt of the event causing the remote device to:
monitor for an interaction with the application presenting the data of the first event, the interaction corresponding to the activity performed by the user, and
generate the response data to send to the at least one server based on the interaction detected on the application presenting the data of the first event.

7. The system of claim 1, wherein the at least one server is further configured to:
identify, from the query responses of the data received from the remote device, a behavioral endpoint for the user and a target date for achieving the behavioral endpoint, and
generate, using the behavioral endpoint and the target date, the event management policy for the user to include the plurality of events.

8. The system of claim 1, wherein the at least one server is further configured to generate, using the profile type for the user, the event delivery schedule for the event management policy, the event delivery schedule specifying a plurality of times for corresponding data of the plurality of events to present at the remote device associated with the user.

9. The system of claim 1, wherein the at least one server is further configured to generate, using location information of the remote device associated with the user, the event management policy to include the plurality of events.

10. The system of claim 1, wherein the at least one server is further configured to generate a new event to include in the plurality of events of the event management policy for the user based on the event identifier, the status identifier, and the delivery timestamp of the record.

11. A method comprising:
establishing, by a server having one or more processors, a communication channel with a remote device associated with a user;
determining, by the server, a profile type for the user based on data received from the remote device of the user, the data including query responses;
generating, by the server, using the profile type for the user, an event management policy to include a plurality of events to be transmitted to the remote device of the user at a corresponding plurality of times in accordance with a delivery schedule defined by the event management policy, each event of the plurality of events specifying one or more activities to be performed by the user associated with the remote device;
transmitting, by the server to the remote device via the communication channel, a first event of the plurality of events of the event management policy in accordance with an event delivery schedule to cause an application executing on the remote device to a first action corresponding to the first event and to record response data corresponding to an activity performed by the user in response to the first action via the application;
receiving, by the server, from the remote device via the communication channel, the response data identifying an event identifier corresponding to the first event, a status identifier indicating the activity performed by the user in response to the first action via the application on the remote device, and a delivery timestamp indicating a time at which the event is provided;
storing, by the server on an activity log for the user maintained in the memory, responsive to the receipt of the response data, a record corresponding to the first event provided to the remote device, the record including the event identifier, the status identifier, and the delivery timestamp;
modifying, by the server, using the event identifier, the status identifier, and the delivery timestamp of the record stored on the activity log generated from the response data from the remote device, the delivery schedule of the event management policy for the user associated with the remote device; and
transmitting, by the server, to the remote device via the communication device, a second event from the plurality of events in accordance with the modified delivery schedule to cause the application executing on the remote device to perform a second action corresponding to the second event to induce a change to at least one action performed by the user in response to the second action corresponding to the second event via the remote device.

12. The method of claim 11, wherein the received data identifies a time duration indicating a length of time taken by the user in performing the activity in response to the presentation of the data of the first event via the remote device, and further comprising and further comprising:
storing by the server, on the activity log, the record including time duration; and
modifying, by the server, at least one of the plurality of events based on the time duration of the record.

13. The method of claim 11, further comprising selecting, by the server, from a plurality of event management policies, the event management policy based on the profile type for the user.

14. The method of claim 11, further comprising receiving, by the server from the remote device, the response data generated by the remote device in response to an interaction with the application on the remote response subsequent to the presentation of the data corresponding to the first event, the first event corresponding to at least one of a controlled breathing event, a social engagement event, a personalized message event, a treatment adherence event, an incentive event, professional caregiving event, and a digital diversion event.

15. The method of claim 11, further comprising modifying, by the server, at least one of the times specified by the event delivery schedule using the event identifier, the status identifier, and the delivery timestamp of the record stored on the activity log.

16. The method of claim 11, further comprising transmitting, by the server, the first event of the plurality of events to the remote device, receipt of the event causing the remote device to:
monitor for an interaction with the application presenting the data of the first event, the interaction corresponding to the activity performed by the user, and
generate the response data to send to the at least one server based on the interaction detected on the application presenting the data of the first event.

17. The method of claim 11, further comprising:
identifying, by the server, from the query responses of the data received from the remote device, a behavioral endpoint for the user and a target date for achieving the behavioral endpoint, and
generating, by the server, using the behavioral endpoint and the target date, the first event management policy for the user to include the plurality of events.

18. The method of claim 11, further comprising generating, by the server, using the profile type for the user, the event delivery schedule for the event management policy, the event delivery schedule specifying a plurality of times for corresponding data of the plurality of events to present at the remote device associated with the user.

19. The method of claim 11, further comprising generating, by the server, using location information of the remote device associated with the user, the event management policy to include the plurality of events.

20. The method of claim 11, further comprising generating, by the server, a new event to include in the plurality of events of the event management policy for the user based on the event identifier, the status identifier, and the delivery timestamp of the record.
